SEQUENCE LISTING (4 sequences)

SEQ ID NO: 1 - DNA, 25 bp, synthetic construct
  source 1..25: mol_type = other DNA
aatcttaacaccttgtacagcccca

SEQ ID NO: 2 - DNA, 21 bp, synthetic construct
  source 1..21: mol_type = other DNA
agtgcctgctccagacttaaa

SEQ ID NO: 3 - RNA, 20 nt, synthetic construct
  source 1..20: mol_type = other RNA
gtgctcctttccaggagtgg

SEQ ID NO: 4 - RNA, 20 nt, synthetic construct
  source 1..20: mol_type = other RNA
acttaaactccacgtcatcg

(19) United States
(12) Patent Application Publication
(10) Pub. No.: US 2026/0284104 A1
(43) Pub. Date: Sep. 24, 2026

(54) SIRPalpha Knockout iPSC-derived and CAR-comprising Macrophages with Enhanced Anti-tumor Activity

(71) Applicant: Wisconsin Alumni Research Foundation, Madison, WI (US)

(72) Inventors: Portia Smith, Madison, WI (US); Jue Zhang, Seattle, WA (US); Igor I. Slukvin, Verona, WI (US)

(21) Appl. No.: 19/382,068

(22) Filed: Nov. 6, 2025

Related U.S. Application Data
(60) Provisional application No. 63/717,093, filed on Nov. 6, 2024.
(60) Provisional application No. 63/746,860, filed on Jan. 17, 2025.

(51) Int. Cl. A61K 35/15; A61K 40/17; A61K 40/31; A61K 40/42; A61P 35/00; C07K 16/30; C12N 5/00; C12N 5/0786; C12N 9/22; C12N 15/11

(52) U.S. Cl. CPC A61K 35/15; A61K 40/17; A61K 40/31; A61K 40/4258; A61P 35/00; C07K 16/3084; C12N 5/0018; C12N 5/0645; C12N 9/226; C12N 15/111; A61K 2239/13; C12N 2310/20; C12N 2500/02; C12N 2500/25; C12N 2500/38; C12N 2500/98; C12N 2500/99; C12N 2501/115; C12N 2501/125; C12N 2501/145; C12N 2501/15; C12N 2501/155; C12N 2501/16; C12N 2501/165; C12N 2501/22; C12N 2501/2303; C12N 2501/2306; C12N 2501/727; C12N 2506/45; C12N 2510/00

(57) ABSTRACT

Provided herein are methods for reducing or eliminating tumor cell growth using genetically engineered macrophages having modified embodiments of signal regulatory protein alpha (SIRPα) gene resulting in reduced or ablated expression thereof, wherein the tumor cell reducing or eliminating activity of the macrophages is not subject to macrophage exhaustion. Therapeutically effective populations of such genetically engineered macrophages are also provided.

### STATEMENT REGARDING FEDERAL SPONSORED RESEARCH

This invention was made with government support under HL142665, OD0101106 and HL134655 awarded by the National Institutes of Health. The government has certain rights in the invention.

### INCORPORATION BY REFERENCE OF SEQUENCE LISTING PROVIDED ELECTRONICALLY

The instant application contains a Sequence Listing that has been submitted electronically and is hereby incorporated by reference in its entirety. The Sequence Listing was created on Nov. 6, 2025, is named “21-1677-US2_SequenceListing.xml”, and is 4,675 bytes in size.

### FIELD OF THE DISCLOSURE

Provided herein are methods for reducing or eliminating tumor cell growth using genetically engineered macrophages having modified embodiments of signal regulatory protein alpha (SIRPα) gene resulting in reduced or ablated expression thereof, wherein the tumor cell reducing or eliminating activity of the macrophages is not subject to macrophage exhaustion. Embodiments of such cells comprising chimeric antigen receptors (CARs) immunologically specific for tumor cells as well as therapeutically effective populations of such genetically engineered macrophages are also provided.

### BACKGROUND

Current cancer immunotherapies concentrate on targeting cancer-specific antigens by the use of chimeric antigen receptors (CAR) T cells or monoclonal antibodies (mAb). There is an urgent need to develop novel immunotherapies that can treat patients with solid tumor cancers. Indiscriminately, solid cancers contain extremely complex tumor microenvironments (TME) that circumvent the innate and adaptive immune systems' anti-tumoral mechanisms. The heterogeneous nature of the tumor and the ability to shield from the immune system are the main reasons why current immunotherapies have not had much success targeting solid cancers. CAR-T cells and mAbs rely on the specificity of the cancer antigen which limits the targeting abilities to mainly circulatory cancers. Other mechanisms such as T cell exclusion, hypoxic milieu, and immunosuppressive myeloid cells create a hostile environment for the adaptive immune system to have any robust anti-tumorigenic effect in other tumor types, particularly solid tumors (Joyce & Fearon (2015) Science (New York, N.Y.), 348(6230), 74-80).

Macrophages are a group of diverse and active immune cells that are found across the body as either tissue-resident macrophages (TR-Macs) or circulating monocyte-derived macrophages (MD-Macs) (Davies, et al., (2013) Nature Immunology, 14(10), 986-995). Developing from the common myeloid progenitor lineage, macrophages are innate antigen presenting immune cells with an incredible appetite for phagocytosis. Upon activation, macrophages polarize into pro-inflammatory M1 subtype, or anti-inflammatory M2 subtype in which there are many other subtypes on a polarization spectrum (Kielbassa et al., (2019) Frontiers in Immunology, 10:2215). Within the TME, tumor cells recruit macrophages via chemokine signaling, such as the CCL2/CCR2 pathway, and polarize the macrophages into a pathological, pro-tumorigenic state called tumor associated macrophages (TAMs) (Chen, et al., (2019) Journal of Biomedical Science, 26(1), 78). Through analyzing the role of macrophages in the TME, it has been found that TAMs mainly resemble M2 macrophages with increased anti-inflammatory and wound-healing signatures (Chanmee, et al., (2014) Cancers, 6(3), 1670-1690). TAMs produce many inhibitory factors including TGF-beta, IL-10, Arg1, IDO, and HIF-1alpha that inhibit and starve the existing T cell population within the TME (Zhu, et al., (2021) Journal of Cancer, 12(1), 54-64). The tumor cells within the TME circumvent the anti-tumorigenic properties of macrophages by the upregulation of immunosuppressive signals, including “don't eat me” cell receptors.

One of the “don't eat me” receptors, CD47, was discovered by Weissman and colleagues as a mechanism for both hematological and solid tumor cancer cells to escape phagocytosis by TAMs (Chao, et al., (2010) Cell, 142(5), 699-713; Majeti, et al., (2009) Cell, 138(2), 286-299; Willingham, et al., (2012) Proceedings of the National Academy of Sciences, 109(17), 6662-6667). This cell surface receptor on tumor cells is recognized by the myeloid-specific receptor: signal regulatory protein alpha (SIRPα). SIRPα is abundantly expressed in macrophages, dendritic cells, and neutrophils. SIRPα is a ligand for the ubiquitously expressed “don't-eat-me” signal molecule CD47. SIRPα also promotes M2 polarization of tumor-associated macrophages. In physiological state, CD47 is expressed ubiquitously on normal, healthy cells as a marker of self, notably red blood cells (RBC) express CD47 to prevent macrophages from phagocytosing them as they mature (Oldenborg, et al., (2000) Science (New York, N.Y.), 288(5473), 2051-2054; Oldenborg, et al., (2001) The Journal of Experimental Medicine, 193(7), 855-862). When CD47 and SIRPα interaction occurs, a signal is transduced to the immunoreceptor tyrosine-based inhibitory motifs (ITIMs) on the cytosolic tail of SIRPα. The ITIMs recruit SUP-1 and SHP-2 domain-containing phosphatases which block Myosin IIa and subsequent cytoskeletal rearrangement (Murata, et al., (2018) Cancer Science, 109(8), 2349-2357). Therefore, CD47/SIRPα activation specifically blocks phagocytosis from occurring when activating phagocytic stimuli is present.

Overexpression of CD47 has been previously correlated to poor prognosis in many hematological cancers (Eladl, E. et al., (2020) Journal of Hematology & Oncology 13, 96; Majeti, R. et al., (2009) Cell 138, 286-299) as well as solid tumors, including breast (Yuan, J. et al., (2019) Oncology Letters 18, 3249-3255; Yuan, J. et al., (2019) Cancer Cell Int 19, 238) ovarian (Willingham, S. B. et al., (2012) PNAS 109, 6662-6667; Li, Y. et al., (2017) Am J Transl Res 9, 2901-2910; Tan, M. et al., (2015) Am J Cancer Res 5, 2777-2787), endometrial (Yang, M., et al., (2022) J Oncol 2022, 7188972), gastric (Yoshida, K. et al., (2015) Cancer Med 4, 1322-1333; Shi, M. et al., (2021) Cancer Immunol Immunother 70, 1831-1840), non-small cell lung cancer (Xu, Y. et al., (2020) ESMO Open 5, e000823), and clear cell renal carcinoma. Due to the vast upregulation of CD47 across many solid tumor cancers, targeting this signaling pathway by knocking out SIRPα in macrophages to generate cellular immunotherapy has the potential for widespread cancer treatment. The blockade of CD47 and SIRPα has been previously explored, however, while it has demonstrated high efficacy against blood cancers, it often require dual-antibody blockade to achieve substantial tumor-killing effects (Chao, M. P. et al., (2010) Cell 142, 699-713; Barkal, A. A. et al., (2019) Nature 572, 392-396; Advani, R. et al., (2018) N Engl J Med 379, 1711-1721), which have not been proven to be clinically effective (Isenberg, J. S. & Montero, E. (2024) Clinical and Translational Medicine 14, e1584). Targeting CD47 therapeutically has proven challenging, inter alia, due to the intricate network of molecular interactions that CD47 participates in.

Macrophages have unique advantages for use cellular immunotherapy due to their access to the TME, phagocytic capabilities, and direct communication to the adaptive immune system via antigen presentation. While macrophages exposed to mAb-opsonized target cells display an initial, rapid burst of anti-body dependent cellular phagocytosis (ADCP), this notable increase is followed by a pronounced decline in phagocytic activity (Pinney, J. J. et al., (2020) Blood 136, 2065-2079). This pronounced decline results in a refractory period that can persist for days, even with subsequent exposure to mAb-opsonized targets. This diminished capacity of ADCP, referred to as hypophagia or “macrophage exhaustion”, impairs macrophage-mediated clearance of mAb-opsonized target cells and potentially can hinder the antitumor activities against solid tumor cancers (Pinney, J. J. et al., (2020)).

Therefore, there exists a need for new immunotherapies that provide for specific targeting of solid tumors expressing CD47 by macrophages which are capable of clinically relevant sustained phagocytic activity that circumvent hypophagia.

### SUMMARY OF THE DISCLOSURE

This application provides methods for reducing or eliminating signal regulatory protein alpha (SIRPα) ligand-expressing tumor cell growth using genetically engineered macrophages having modified embodiments of the SIRPA gene resulting in reduced or ablated expression thereof, wherein the tumor cell reducing or eliminating activity of the macrophages is characterized by reduced macrophage exhaustion thereby. The current disclosure also provides methods of treating a subject bearing a SIRPα ligand-expressing tumor using genetically engineered macrophages having modified embodiments of the SIRPA gene resulting in reduced or ablated expression thereof, wherein the tumor cell reducing or eliminating activity of the macrophages is characterized by reduced macrophage exhaustion thereby.

In one aspect, the disclosure provides a method of reducing or eliminating growth of SIRPα ligand-expressing tumor cells by contacting the tumor cells with an effective amount of a combination of SIRPA-KO macrophages and an antibody immunologically specific for the tumor cell. Further provided herein is a method of reducing or eliminating growth of SIRPα ligand-expressing tumor cells by contacting the tumor cells with an effective amount of SIRPA-KO macrophages expressing a chimeric antigen receptor (CAR) that is immunologically specific or the tumor cell.

In another aspect, the disclosure provides a method of treating a subject bearing a SIRPα ligand-expressing tumor by contacting the tumor with an effective amount of a combination of SIRPA-KO macrophages and an antibody immunologically specific for the tumor cell. Further provided herein is a method of treating a subject bearing SIRPα ligand-expressing tumor by contacting the tumor with an effective amount of SIRPA-KO macrophages expressing a chimeric antigen receptor (CAR) that is immunologically specific or the tumor cell.

In some embodiments of the disclosure, the SIRPα ligand expressed by the tumor cells is CD-47.

In some embodiments of the disclosure, the modified macrophages are produced from pluripotent stem cells by a method comprising:
(a) culturing human pluripotent stem cells having inhibited expression of signal regulatory protein alpha (SIRPα) in normoxic conditions for about 24 hours in serum-free culture medium comprising of L-ascorbic acid-2-phosphate magnesium, sodium selenium, transferrin, insulin, NaHCO₃, fibroblast growth factor 2 (FGF2), transforming growth factor beta 1 (TGFβ1), and a Rho kinase (ROCK) inhibitor;
(b) further culturing the human pluripotent stem cells of (a) in hypoxia conditions for about 48 hours in serum-free culture medium comprising bone morphogenetic protein 4 (BMP4), FGF2, Activin A, an inhibitor of glycogen synthase 3 (GSK3), and a ROCK inhibitor to induce mesoderm formation;
(c) further culturing the cultured cells of (b) in hypoxic conditions for about 48 hours in serum-free culture medium comprising FGF2, a vascular endothelium growth factor (VEGF), and an inhibitor of TGFβ-mediated signaling to induce hemogenic endothelium formation;
(d) further culturing the cultured cells of (c) in normoxic conditions for about 6 days in serum-free culture medium comprising FGF2, a VEGF, stem cell factor (SCF), thrombopoietin (TPO), interleukin-6 (IL-6), and interleukin-3 (IL-3), wherein the hemogenic endothelium differentiate into HPCs;
(e) culturing the HPCs of (d) in normoxic conditions for about 6 days in serum-free culture medium comprising macrophage colony-stimulating factor (M-CSF), IL-3, and IL-6 to obtain myeloid progenitors and monocytic cells; and
(f) further culturing the cultured cells of (e) in normoxic conditions for about 4 days in serum-free culture medium comprising M-CSF, whereby the cultured myeloid progenitors and monocytes differentiate into a cell population comprising modified macrophages.

In particular embodiments the inhibitor of TGFβ-mediated signaling is SB431542. In other particular embodiments the inhibitor of GSK3 is lithium chloride (LiCl). In certain embodiments the ROCK inhibitor used in the practice of the methods disclosed herein is Y-27632.

Advantageously expression of SIRPα is inhibited in the human pluripotent stem cells by gene mutation, RNA-mediated inhibition, RNA editing, DNA gene editing or base editing. Particular gene editing methods useful in the practice of these methods use a nuclease that includes but are not limited to a meganuclease, zinc-finger nucleases (ZFNs), transcription activator-like effector nucleases (TALENs), and Cas enzyme, particularly embodiments using Cas9. Advantageously gene editing results in knocking out SIRPα expression.

In certain embodiments the pluripotent stem cells are induced pluripotent stem cells (iPSCs).

Also provided herein are modified macrophages produced according to the disclosed methods.

In another aspect, the disclosure provides a method of producing modified macrophages from pluripotent stem cells, the method comprising:
(a) transiently introducing exogenous ETV2 in human pluripotent stem cells having inhibited expression of SIRPα and culturing the ETV2-induced pluripotent stem cells in serum-free culture medium comprising FGF-2 to produce a population of ETV2-induced hematoendothelial progenitor cells (ETV2-induced HEPs);
(b) culturing the ETV2-induced HEPs in serum-free and xeno-free culture medium comprising granulocyte-macrophage colony-stimulating factor (GM-CSF) and FGF2 for a sufficient time to produce non-adherent myeloid progenitors;
(c) culturing the non-adherent myeloid progenitors in serum-free and xeno-free culture medium comprising M-CSF, IL-6, and IL-3; and
(d) further culturing the cultured cells of (c) in serum-free and xeno-free culture medium comprising M-CSF for a sufficient time to differentiate the non-adherent myeloid progenitors into modified macrophages.

In particular embodiments the serum-free and xeno-free culture medium in step (b) further comprises UM171.

Advantageously expression of SIRPα is inhibited in the human pluripotent stem cells by gene mutation, RNA-mediated inhibition, RNA editing, DNA gene editing or base editing. Particular gene editing methods useful in the practice of these methods use a nuclease that includes but are not limited to a meganuclease, zinc-finger nucleases (ZFNs), transcription activator-like effector nucleases (TALENs), and Cas enzyme, particularly embodiments using Cas9. Advantageously gene editing results in knocking out SIRPα expression.

In particular embodiments, step (a) of the method comprises culturing the ETV2-induced pluripotent stem cells for about 1-2 days; step (b) of the method comprises culturing the ETV2-induced HEPs for about 6-7 days; and step (c) and step (d) comprises culturing myeloid progenitor cells for about 9 to 10 days.

Also provided herein are modified macrophages produced according to these disclosed methods.

In particular embodiments, the modified macrophages produced according to these disclosed methods have a phenotype exhibiting a heightened capacity for antibody dependent cellular phagocytosis.

In another aspect, the disclosure provides for a homogenous population of modified macrophages, wherein the modified macrophages are SIRPA knockout macrophages with a tumor-killing capacity phenotype exhibiting heightened capacity for antibody dependent cellular phagocytosis without experiencing hypophagia.

Also provided herein are methods for treating cancer in a by administering to a patient in need thereof a therapeutically effective amount of the modified macrophages produced by the methods disclosed herein.

In particular embodiments, the SIRPα-KO macrophages express an anti-disialoganglioside GD2 (GD2) chimeric antigen receptor (CAR).

In particular embodiments, the tumor cell is, but not limited to, a neuroblastoma, retinoblastoma, medulloblastoma, glioblastoma, melanoma, lung cancer, pancreatic cancer, bladder cancer, colorectal cancer, sarcoma, or breast cancer cell.

Also provided herein is a pharmaceutical composition comprising a population of SIRPα-KO modified macrophages as disclosed herein.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

FIG. 1A depicts the mechanisms by which tumor cells within the TME circumvent the anti-tumorigenic properties of macrophages by the upregulation of immunosuppressive signals, including “don't eat me” cell receptors, and the mechanisms by which genetically engineered iMacs described within overcome the upregulation of immunosuppressive signals by both mAb-driven and CAR-driven anti-tumor mechanisms.

FIG. 1B-1L show the generation of SIRPα-Knockout (KO) induced pluripotent stem cells (iPSCs) and the subsequent differentiation of said cells into macrophages

FIG. 1B is a schematic of CRISPR/Cas9 driven knockout of SIRPA gene at exon 3 using two sgRNAs.

FIG. 1C shows DNA extractions for genomic PCR on an agar gel for each clone. Specifically, nucleofection of hiPSCs with sgRNAs and Cas9 protein was performed. After several days, clones were selected and expanded.

FIG. 1D shows phase-contrast images of WT and SIRPA-KO iPSCs.

FIG. 1E shows a 2D monolayer differentiation schematic for generation of iPSC-Macrophages.

FIG. 1F shows phase contrast microscopy images of WT and KO cell cultures undergoing endothelial-to-hematopoietic transition and HP formation during days 6 to 9 of culture.

FIG. 1G shows expression of CD43, CD235a, CD41a, and CD45 in WT and SIRPA-KO day 9 floating HPs analyzed in flow cytometry.

FIG. 1H shows the yield of HPs from WT and SIRPA-KO cultures on day 9 of differentiation. Data are represented as mean±SEM (WT n=27, KO n=17). ****p<0.0001, Welch's t test.

FIG. 1I shows a schematic for generation of macrophages from floating HPs.

FIG. 1J shows morphology of WT and SIRPA-KO iPSC-derived macrophages (iMacs). Cells were stained with Wright-Giesma and imaged using brightfield microscopy.

FIG. 1K shows a western blot of WT and SIRPA-KO iMacs where glyceraldehyde-3-phosphate dehydrogenase (GAPDH) was used as a loading control. FIG. 1L shows expression of CD14, CD163, CD206, CD86, CD80, and HLA-DR in WT and SIRPA-KO iMacs stimulated with IFN-gamma+LPS (M1), IL-4 (M2), or unstimulated (M0) Unstained (Uns.) macrophages were used as a control. The analysis is representative of n=3.

FIG. 2A-J show the SIRPA-KO iMacs have superior antibody-dependent antitumor capacity in vitro.

FIG. 2A shows phagocytosis by WT or SIRPA-KO iMacs by fluorescent microscopy. iMacs were stained with Cell Trace Violet, then co-cultured with WM-266-4 GFP+Luc2+ cancer cells+/−anti-GD3 in a 1:1 effector-to-target ratio. After 5 hours, cytospins were made.

FIG. 2B-D show quantification of ADCP using flow cytometry. FIG. 2B-C iMacs were cultured with SK-OV-3 GFP-Luc2+ cancer cells+/−anti-HER2 for 24 hours or FIG. 2D WM-266-4 GFP-Luc2+ cancer cells+/−anti-GD3 for 6 hours at indicated effector-to-target ratio.

FIG. 2E-F shows a luciferase-based assay used to quantify ADCC. WT or SIRPA-KO iMacs were co-cultured with (2E) SK-OV-3 GFP-Luc2+ cancer cells+/−anti-HER2 or (2F) WM-266-4 GFP-Luc2+ cancer cells+/−anti-GD3 in indicated E:T ratio. (2E-F) Results are mean±SEM (n=3), *p=0.0488, **p=0.0014, ***p=0.0005, ****p<0.0001, 2way ANOVA. FIG. 2G-H show the time kinetics of ADCC. FIG. 2G shows results that are mean±SEM (n=6); *p=0.0153, **p=0.0013, ****p<0.001, 2way ANOVA.

FIG. 2H shows GFP+SK-OV-3 cancer cell viability in cultures with iMacs and mAb were taken via fluorescence microscopy. FIG. 2I shows a schematic representation of the of the SIRPA knock-in (KI) molecule construct for the AAVS1 safe harbor locus.

FIG. 2J shows an ADCC assay demonstrating restoration of the checkpoint response following expression of SIRPα in SIRPA-KO cells. Results are mean±SEM (n=3); ****p<0.0001, 2way ANOVA.

FIG. 3A-B show knocking out SIRPA does not globally alter the transcriptome of macrophages.

FIG. 3A shows a schematic of preparation of WT and SIRPA-KO iMacs for bulk RNA-sequencing.

FIG. 3B shows a volcano plot of WT and SIRPA-KO were either cultured alone (Group 1), with SK-OV-3 and anti-HER2 for 24 hours (Group 2), or with SK-OV-3 and anti-HER2 for 96 hours (Group 3).

FIG. 4A-I show SIRPα iMacs resist mAb-driven macrophage exhaustion during cancer re-challenges.

FIG. 4A shows a schematic for in vitro cancer re-challenge assay. WT and SIRPA-KO iMacs were co-cultured with AK-OV-3 GFP-Luc2+ cancer cells+anti-HER2 at a 10:1 and 20:1 effector-to-target ratio.

FIG. 4B shows a luciferase assay used to detect ADCC during in vitro cancer re-challenge assay. Results are mean±SEM (n=6); **p=0.0034, ***p<0.001, ****p<0.0001, 2way ANOVA.

FIG. 4C shows fluorescent microscopy images of GFP+SK-OV-3 viable cells during and in vitro cancer re-challenge assay.

FIG. 4D shows a schematic for 2-hour phagocytosis challenge with GFP-Luc2 SK-OV-3 post 96-hour serial exposure to unmodified SK-OV-3

FIG. 4E shows the results of WT and SIRPA-KO iMacs subject to a 2-hour phagocytosis assay with fresh GFP-expressing SK-OV-3 and anti-HER2 mAb.

FIG. 4F shows the results by percentage (%) of GFP-expressing iMacs within the CD45+-gated cells; results are mean±SEM (n=2); **p<0.01, student's paired t-test.

FIG. 4G shows WT and SIRPA-KO iMacs subject to an in vitro serial tumor exposure assay as described in FIG. 4A for 96 hours, with or without anti-HER2 or SK-OV-3 target cells, then isolated for flow cytometric analysis of CD16, CD32, and CD64 expression in CD45+-gated cells. All Geometric MFI values of WT and SIRPA-KO iMacs were normalized to their respective WT or SIRPA-KO iMacs alone at 0 hours. Results are mean±SEM (n=3); *p<0.05, **p<0.01, ***p<0.001, ****p<0.0001, two-way ANOVA.

FIG. 4H shows WT and SIRPA-KO iMacs subject to serial exposure to unmodified SK-OV-3 tumor cells as described in FIG. 4A, then at 96 hours, total cell culture was collected and re-plated for DQ-Ovalbumin challenge for 30 minutes in 37° C. incubator. Cells incubated at 4° C. (4C) were used as a control for background fluorescence. Total cells were then isolated and analyzed by flow cytometry. OVA proteolysis, depicted as % DQ-OVA+, was evaluated after gating CD45+ iMacs by flow cytometry. Results are mean±SEM (n=2); one-way ANOVA.

FIG. 4I shows secretome analysis of macrophages after cancer re-challenge assay. WT and SIRPA-KO iMacs were co-cultured with SK-OV-3 GFP-Luc2+ cancer cells+/−anti-HER2 at a 20:1 effector to target ratio. Every 24 hours, total media was replenished with fresh SK-OV-3+/−anti-HER without disturbing the existing co-culture. At 96 hours, Cell culture media of the co-cultures were collected for secretome analysis. Results are mean±SEM (n=3); *p<0.05, **p<0.01, ***p<0.001, ****p<0.0001, student's t test.

FIG. 5A-H show SIRPA-KO iMacs+anti-HER2 exhibited little efficacy against an in vivo SK-OV-3 tumor model.

FIG. 5A shows a schematic of in vivo SK-OV-3 tumor model establishment. Female NSG mice were engrafted with 7.5×10⁵ SK-OV-3 GFP-Luc2+ cancer cells via IP injection and five days later, treated with either 100 ug aHER2 alone, or with 7×10⁶ WT or SIRPA-KO iMacs via IP injection.

FIG. 5B shows bioluminescent images of tumor xenografts over time for each treatment group.

FIG. 5C shows quantification of SK-OV-3 tumor xenografts over time for each treatment group. Results are mean total flux (photons/s)±SEM (NC n=2, PBS n=4, aHER2 n=4, WT+aHER2 n=4, KO+aHER2 n=4); **p=0.002, ***p<0.001, 2way ANOVA.

FIG. 5D shows Kaplan-Meier survival analysis of mice for each treatment group from FIG. 5C as analyzed using the log-rank test: *p=0.0169, **p=0.0069.

FIG. 5E shows a schematic of in vivo SK-OV-3 tumor model establishment. Female NSG mice were engrafted with 7.5×10⁵ SK-OV-3 GFP-Luc2+ cancer cells via IP injection and five days later, treated with either 50 ug aHER2 alone via IP injection, or with 5×10⁶ WT or SIRPA-KO iMacs via IP injection. On day 14, an additional 50 ug aHER2 was given via IP injection.

FIG. 5F shows bioluminescent images of tumor xenografts over time for each treatment group. NC, negative control.

FIG. 5G shows quantification of SK-OV-3 tumor xenografts over time for each treatment group. NC, negative control. Results are mean total flux (photons/s)±SEM (NC n=2, PBS n=8, aHER2 n=5, WT+aHER2 n=5, KO+aHER2 n=3); ****p<0.0001, two-way ANOVA.

FIG. 5H shows Kaplan-Meier survival analysis of mice for each treatment group from FIG. 5G as analyzed using the log-rank test. **p=0.0066, ***p=0.0007.

FIG. 6A-I show ablating SIRPA potentiates the tumor-killing capacity of anti-GD2-CAR macrophages in vitro.

FIG. 6A shows a schematic of generation of anti-GD2-CAR (CAR) iMacs and anti-GD2 CAR SIRPA-KO (CS) iMacs.

FIG. 6B shows CAR and CS iMacs stained for DAPI (blue) and anti-SIRPα (green) and subject to fluorescence microscopy.

FIG. 6C shows a cytotoxicity assay used to quantify antitumor capacity in vitro where CAR and CS iMacs were co-cultured with CHLA-136 GFP-Luc2+ neuroblastoma cells.

FIG. 6D shows a cytotoxicity assay used to quantify antitumor capacity in vitro where CAR and CS iMacs were co-cultured with SKOV3 GFP-Luc2+ cancer cells at indicated effector-to-target ratios for 24 hours. Data are mean±SEM (CHLA-136 n=6, SK-OV-3 n=3); *p<0.05, ****p<0.0001, 2way ANOVA.

FIG. 6E shows a cytotoxicity assay used to quantify in vitro tumor viability over time where CAR or CS iMacs were co-cultured with CHLA-136 GFP-Luc2+ neuroblastoma cells.

FIG. 6F shows secretome analysis of iMacs after co-culture with CHLA-136. CAR and CS iMacs were co-cultured with either cultured alone or with CHLA-136 GFP-Luc2+ cancer cells at a 10:1 effector-to-target ratio. After 24 hours, cell culture medium was collected for secretome analysis. Results are mean±SEM (n=3); *p<0.05, **p<0.01, ***p<0.001, ****p<0.0001, student's t test.

FIG. 6G shows a schematic for the in vitro re-challenge assay. CAR and CS iMacs were co-cultured with CHLA-136 GFP-Luc2+ cancer cells at an initial 10:1 E:T ratio.

FIG. 6H shows a luciferase assay used to detect cytotoxicity during in vitro cancer re-challenge assay. Results are mean±SEM (n=6); ****p<0.0001, two-way ANOVA.

FIG. 6I shows fluorescent microscopy images of GFP+ GHLA-136 viable cells during in vitro cancer re-challenge assay.

FIG. 7A-D show ablating SIRPα in GD2-CAR-iMacs reverses CAR-mediated exhaustion.

FIG. 7A shows a schematic for 2-hour phagocytosis challenge with GFP-Luc2 CHLA-136 tumors post 96-hour serial exposure to unmodified CHLA-136.

FIG. 7B shows a representative dot plot showing phagocytosis of GFP-expressing CHLA-136 by CD45⁺-gated CAR-iMacs. Results are mean±SEM (n=6 from two independent experiments); **p<0.0031, student's paired t-test.

FIG. 7C shows a bar graph showing phagocytosis of GFP-expressing CHLA-136 by CD45⁺-gated CAR-iMacs. Results are mean±SEM (n=6 from two independent experiments); **p<0.0031, student's paired t-test.

FIG. 7D shows surface expression of GD2-CAR 96 hours after serial exposure to CHLA-136. All Geometric MFI values of CAR iMacs were calculated on CD45⁺ population and normalized to their respective GD2-CAR or GD2-SIRPA-KO-CAR iMacs alone at 0 hours. Results are mean±SEM (n=3); ****p<0.0001, two-way ANOVA.

FIG. 8A-C demonstrate that GD2-SIRPA-KO-CAR-iMacs delay tumor growth of highly metastatic neuroblastoma-engrafted mice.

FIG. 8A shows a schematic of in vivo CHLA-136 tumor model establishment. Male and female NCG-x mice were engrafted with 6×10⁵ CHLA-136 GFP-Luc2+ cancer cells via IV injection and two days later, treated with 5×10⁶ CAR or CS iMacs via IV injections on days 0, 3, and 6.

FIG. 8B shows bioluminescent images of tumor xenografts over time for each treatment group. NC, negative control.

FIG. 8C shows quantification of CHLA-136 tumor xenografts over time for each treatment group. NC, negative control. Results are mean total flux (photons/s)±SEM (n=7); **p=0.002, ***p<0.001, two-way ANOVA.

### DETAILED DESCRIPTION

The disclosure generally relates to methods for reducing or eliminating growth of SIRPα ligand-expressing tumor cells by contacting tumor cells with an effective amount of SIRPA-KO macrophages in combination with an antibody immunologically specific for the tumor cell, or where the SIRPA-KO macrophage expresses a chimeric antigen receptor (CAR) that is immunologically specific for the tumor cell.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

Throughout this specification, unless the context specifically indicates otherwise, the terms “comprise” and “include” and variations thereof (e.g., “comprises,” “comprising,” “includes,” and “including”) are understood to indicate the inclusion of a stated component, feature, element, or step or group of components, features, elements or steps but not the exclusion of any other component, feature, element, or step or group of components, features, elements, or steps. Any of the terms “comprising”, “consisting essentially of”, and “consisting of” can be replaced with either of the other two terms, while retaining their ordinary meanings

As used herein, the singular forms “a,” “an,” and “the” include plural referents unless the context clearly indicates otherwise.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein and in the drawings, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Subject” or “patient” as used herein are used interchangeably and refer to a warm-blooded animal such as a mammal, preferably a human, which is afflicted with, or has the potential to be afflicted with a tumor as described herein.

“Express” or “expression” as used herein refers to transcription and translation of a nucleic acid coding sequence resulting in production of the encoded polypeptide.

“Pluripotent stem cells” refer to cells having the capacity to differentiate into cells of all three germ layers. “Embryonic stem cells” or “ESCs” mean a pluripotent cell or population of pluripotent cells derived from an inner cell mass of a blastocyst. See Thomson et al., (1998) Science 282:1145-1147.

“Induced pluripotent stem cells” or “iPS cells” refer to a pluripotent cell or population of pluripotent cells that may vary with respect to their differentiated somatic cell of origin, that may vary with respect to a specific set of potency-determining factors and that may vary with respect to culture conditions used to isolate them, but nonetheless are substantially genetically identical to their respective differentiated somatic cell of origin and display characteristics similar to higher potency cells, such as ESCs. See, e.g., Yu et al., (2007) Science 318:1917-1920.

“Hematopoietic precursor cells (HPCs)” and “hematopoietic progenitors (HPs)” refer to immature multipotent progenitor cells of the hematopoietic lineage. HPCs are characterized by surface expression of CD45 and, in some cases, CD34, and a capacity to differentiate into myeloid and lymphoid progenitors and terminally differentiated lymphoid and myeloid cells.

“Myeloid progenitors” are cells capable of differentiating into cell types of the myeloid lineages.

“Chemically-defined culture” “fully defined, growth factor free culture conditions,” and “fully-defined conditions” indicate that the identity and quantity of each medium ingredient is known and the identity and quantity of supportive surface is known.

“Xeno-free culture medium” refers to medium that does not contain any components derived from animal sources, such as, for example, serum.

As used herein, a “SIRPA knockout” or “SIRPA-KO” is intended to encompass any disruption or deletion of SIRPA gene that results in the dysfunction or nonfunction of the SIRPα protein as it pertains to binding to CD47 or other SIRPα binding partners, such as surfactant protein A, and protein tyrosine phosphatases SUP-1 and SHP-2, and/or as it pertains to the signaling within the SIRPα-expressing cell. See Barclay, A. N & Brown, M. H., (2006) Nat Rev Immunol 6, 457-464; Murata, Y. et al., (2014) The Journal of Biochemistry 155(6), 335-344. This definition includes the insertion of an external plasmid/gene within the SIRPA gene locus or deleting a portion or the entire region of the SIRPA gene.

“SIRPα ligand-expressing tumor cells” and “SIRPα ligand-expressing tumors” as used herein refers to tumor cells and tumors that express any binding partner of SIRPα. This may include, but is not limited to, CD47, surfactant protein A, and protein tyrosine phosphatases SUP-1 and SHP-2. See Barclay, A. N & Brown, M. H., (2006); Murata, Y. et al., (2014).

### Methods for Production of Macrophages Using Morphogen-Driven Differentiation Systems

The methods provided herein comprise differentiating human pluripotent stem cells under conditions that promote differentiation of the pluripotent stem cells into hematopoietic progenitor cells and macrophages.

In some embodiments provided herein is a method of producing modified macrophages from pluripotent stem cells, the method comprising:
(a) culturing human pluripotent stem cells having inhibited expression of signal regulatory protein alpha (SIRPα) in normoxic conditions for about 24 hours in a serum-free culture medium comprising of L-ascorbic acid-2-phosphate magnesium, sodium selenium, transferrin, insulin, NaHCO₃, fibroblast growth factor 2 (FGF2), transforming growth factor beta 1 (TGFβ1), and a Rho kinase (ROCK) inhibitor;
(b) further culturing the human pluripotent stem cells of (a) in hypoxia conditions for about 48 hours in a serum-free culture medium comprising bone morphogenetic protein 4 (BMP4), FGF2, Activin A, an inhibitor of glycogen synthase 3 (GSK3), and a ROCK inhibitor to induce mesoderm formation;
(c) further culturing the cultured cells of (b) in hypoxic conditions for about 48 hours in a serum-free culture medium comprising FGF2, a vascular endothelium growth factor (VEGF), and an inhibitor of TGFβ-mediated signaling to induce hemogenic endothelium formation;
(d) further culturing the cultured cells of (c) in normoxic conditions for about 6 days in a serum-free culture medium comprising FGF2, a VEGF, stem cell factor (SCF), thrombopoietin (TPO), interleukin-6 (IL-6), and interleukin-3 (IL-3), wherein the hemogenic endothelium generates hematopoietic precursor cells (HPCs);
(e) culturing the HPCs of (d) in normoxic conditions for about 6 days in a serum-free culture medium comprising macrophage colony-stimulating factor (M-CSF), IL-3, and IL-6 to generate myeloid progenitors and monocytes; and
(f) further culturing the cultured cells of (e) in normoxic conditions for about 4 days in a serum-free culture medium comprising M-CSF, whereby the cultured myeloid progenitors and monocytes differentiate into a cell population comprising modified macrophages.

Suitable pluripotent cells for use herein include human embryonic stem cells (hESCs) and human induced pluripotent stem (iPS) cells. ESCs are commercially available from sources such as WiCell Research Institute (Madison, Wis.). In particular embodiments, the pluripotent stem cells are induced pluripotent stem cells.

The pluripotent stem cells used in the methods disclosed herein have inhibited expression of SIRPα. SIRPα is abundantly expressed in macrophages, dendritic cells, and neutrophils. SIRPα is a ligand for the ubiquitously expressed “don't-eat-me” signal molecule CD47, surfactant protein A, and binds to protein tyrosine phosphatases SUP-1 and SHP-2. See Barclay, A. N & Brown, M. H., (2006); Murata, Y. et al., (2014). SIRPα also promotes M2 polarization of tumor-associated macrophages.

The term “genetically engineered” as used herein refers to cells that have been manipulated using biotechnology to change the genetic makeup of the cells, including the transfer of genes within and across species boundaries to produce improved or non-naturally occurring cells. A human pluripotent stem cell or macrophage that contains an exogenous, recombinant, synthetic, and/or otherwise modified polynucleotide is considered to be a genetically engineered cell and, thus, non-naturally occurring relative to any naturally occurring counterpart. In some cases, genetically engineered cells contain one or more recombinant nucleic acids. In other cases, genetically engineered cells contain one or more synthetic or genetically engineered nucleic acids (e.g., a nucleic acid containing at least one artificially created insertion, deletion, inversion, or substitution relative to the sequence found in its naturally occurring counterpart). Procedures for producing genetically engineered cells are generally known in the art, for example, as described in Sambrook et al., Molecular Cloning, A Laboratory Manual (Fourth Edition), Cold Spring Harbor Press, Cold Spring Harbor, N.Y. (2012) and Doudna et al., CRISPR-Cas, A Laboratory Manual, Cold Spring Harbor Press, Cold Spring Harbor, N.Y. (2016).

“Having inhibited expression of SIRPα,” indicates the gene is repressed or not expressed in a functional protein form. In particular embodiments, the expression of SIRPα is knocked out such that there is no expression of SIRPα. This inhibition or knockout can be obtained by gene mutation, RNA-mediated inhibition, RNA editing, DNA gene editing or base editing.

In particular embodiments, the gene editing method comprises the use of a nuclease selected from a meganuclease, zinc-finger nucleases (ZFNs), transcription activator-like effector nucleases (TALENs), and Cas enzyme. In particular embodiments, the nuclease is a Cas9 enzyme.

In some embodiments, normoxic conditions refer to conditions where oxygen is provided at or about standard atmospheric levels. In some embodiments, normoxic conditions refer to oxygen conditions of about 15% to about 20% oxygen (e.g., about 15%, 16%, 17%, 18%, 19%, 20% O₂).

In some embodiments, hypoxic conditions refer to a level of environmental oxygen (e.g., a cell culture incubator gas mixture) of about 3% O₂ to about 10% O₂. In some embodiments, hypoxic conditions are about 5% 02.

In some embodiments, the culture is “serum-free” which refers to cell culture materials that are free of serum obtained from animal or human (e.g., fetal bovine) blood.

In some embodiments, the culture conditions are feeder-free, meaning that the culture does not use feeder cells. In particular embodiments, the culture conditions are serum-free and feeder-free.

In particular embodiments, the method disclosed herein comprises an attachment step comprising culturing human pluripotent stem cells in a culture medium in normoxic conditions (i.e., where oxygen is provided at or about standard atmospheric levels) for about 24 hours. In particular embodiments, the culture medium is E8-TeSR. “E8 culture medium” and “E8” are used interchangeably and refer to the chemically defined culture medium having the following defined components: DMEM/F12, L-ascorbic acid-2-phosphate magnesium, sodium selenium, and NaHCO₃, transferrin, insulin, FGF2 and TGFβ1.

In some embodiments, the culture medium used for culturing hPSCs to HPCs and/or myeloid progenitors to macrophages is “IF9S” media, which comprises IMDM/F12, L-ascorbic acid 2-phosphate Mg²⁺ salt, monothioglycerol, sodium selenite, polyvinyl alcohol, Glutamax™ non-essential amino acids (NEAA), chemically defined lipid concentrate (Life Technologies; Cat. No. 1905031), Holo-Transferrin, and insulin.

In particular embodiments, the culture medium comprises a Rho kinase (ROCK) inhibitor. Rock inhibitors, which are known in the art and include, but are not limited to, for example, Y27632 (commercially available from Stem Cell Technologies), and those found in Liao J K, Seto M, Noma K. Rho kinase (ROCK) inhibitors. J Cardiovasc Pharmacol. 2007; 50(1):17-24, the contents of which are incorporated by reference in its entirety.

In some embodiments, the inhibitor of TGFβ-mediated signaling is SB431542. In some embodiments, the inhibitor of GSK3 is lithium chloride (LiCl).

Also provided herein is a population of modified macrophages produced by the methods disclosed herein. The modified macrophages have inhibited expression of SIRPα as well as superior anti-tumor activity for therapeutic purposes.

Uses of Macrophages Using ETV2 Modified mRNA Differentiation Systems

The present disclosure also provides methods for efficient macrophage production from pluripotent stem cells using direct programming with transient expression of ETV2, e.g., by addition of modified mRNA (mmRNA) of ETV2 into the hiPSCs. Initially, hiPSCs are directly programmed into hematoendothelial progenitors using ETV2 mmRNA which transiently produced ETV2 within the cells. Next, the hematoendothelial progenitors are then differentiated into myeloid progenitors in the presence of GM-CSF, FGF2 and optionally UM171 (the presence of UM171 in combination with GM-CSF and FGF2 increases the number of macrophages produced by the methods). Myeloid progenitors which are non-adherent could be continuously collected from cultures every 8-10 days for up to 30 days of post ETV2 transfection. Finally, these myeloid progenitors are subsequently differentiated into macrophages. The methods for macrophage production from pluripotent stem cells using direct programming with transient expression of ETV 2 are described in U.S. Publication No. 20200385676, the contents of which are incorporated by reference in its entirety.

In particular embodiments disclosed herein is a method of producing modified macrophages from pluripotent stem cells, the method comprising:
(a) transiently introducing exogenous ETV2 in human pluripotent stem cells having inhibited expression of SIRPα and culturing the ETV2-induced pluripotent stem cells in serum-free and xeno-free culture medium comprising FGF-2 to produce a population of ETV2-induced hematoendothelial progenitor cells (ETV2-induced HEPs);
(b) culturing the ETV2-induced HEPs in serum-free and xeno-free culture medium comprising granulocyte-macrophage colony-stimulating factor (GM-CSF) and FGF2 for a sufficient time to produce non-adherent myeloid progenitors;
(c) culturing the non-adherent myeloid progenitors in serum-free and xeno-free culture medium comprising M-CSF, IL-6, and IL-3; and
(d) further culturing the cultured cells of (c) in serum-free and xeno-free culture medium comprising M-CSF for a sufficient time to differentiate the non-adherent myeloid progenitors into modified macrophages.

Suitable pluripotent cells for use herein include human embryonic stem cells (hESCs) and human induced pluripotent stem (iPS) cells. ESCs are commercially available from sources such as WiCell Research Institute (Madison, Wis.). In particular embodiments, the pluripotent stem cells are induced pluripotent stem cells.

The pluripotent stem cells used in the methods disclosed herein have inhibited expression of SIRPα.

“Having inhibited expression of SIRPα,” indicates the gene is repressed or not expressed in a functional protein form. In particular embodiments, the expression of SIRPα is knocked out such that there is no expression of SIRPα. This inhibition or knockout can be obtained by gene mutation, RNA-mediated inhibition, RNA editing, DNA gene editing or base editing.

In particular embodiments, the gene editing method comprises the use of a nuclease selected from a meganuclease, zinc-finger nucleases (ZFNs), transcription activator-like effector nucleases (TALENs), and Cas enzyme. In particular embodiments, the nuclease is a Cas9 enzyme.

ETV2 can be transiently introduced into the PSCs by methods known in the art. Methods of transiently expressing ETV2 in PSCs are known in the art, and include, but are not limited to, for example, introducing transiently exogenous nucleic acids encoding the protein of interest (e.g., by plasmid expression vector transfection, or modified mRNA transfection); protein transduction, among others. In one embodiment, mmRNA of ETV-2 (e.g., Accession No: NM_014209.2; SEQ ID NO:4) is introduced into the PSCs by suitable methods. Methods of transiently expressing ETV2 in PSCs are described in U.S. Pat. No. 9,382,531, the contents of which are incorporated by reference in its entirety. Methods of introducing mmRNA into PSCs are known in the art, and include, but are not limited to, the method described in the Examples, for example, by transfection or electroporation. The methods of introducing mmRNA or DNA to transiently express ETV-2 protein is within the skill of one in the art and are not limited to what is demonstrated in the examples

After initiating transient expression of ETV2 in the hPSCs, these cells are cultured for a sufficient time to produce a population of ETV2-induced hematoendothelial progenitor cells (ETV2-induced HEPs). In particular embodiments, a sufficient time is a period of about 24 hours to about 4 days. In some embodiments, a sufficient amount of time to produce a population of ETV2-induced hematoendothelial progenitor cells comprises culturing the ETV2-induced cells for about 1-2 days. In some embodiments, a sufficient amount of time to produce a population of ETV2-induced hematoendothelial progenitor cells comprises culturing the ETV2-induced cells for about 3-8 days, for example, for about 4 days. For example, in some embodiments, the step to produce a population of ETV2-induced hematoendothelial progenitor cells comprises culturing for 3 days, alternatively 4 days, alternatively 5 days, alternatively 6 days, alternatively 7 days, alternatively 8 days to produce ETV2-induced hematoendothelial progenitor cells.

In some embodiments, the culture medium is “serum-free” which refers to cell culture materials that are free of serum obtained from animal or human (e.g., fetal bovine) blood.

In some embodiments, the culture conditions are feeder-free, meaning that the conditions do not use feeder cells. In particular embodiments, the culture conditions are serum-free and feeder-free.

In particular embodiments, the methods disclosed herein use a maintenance culture medium for culturing the hPSCs after transfection with ETV2 mmRNA. In particular embodiments the culture medium is E8-TeSR. E8 culture medium” and “E8” are used interchangeably and refer to the chemically defined culture medium having the following defined components: DMEM/F12, L-ascorbic acid-2-phosphate magnesium, sodium selenium, and NaHCO₃ in a final volume of 200, transferrin, insulin, FGF2 and TGFβ1.

In some embodiments, the culture medium is a xeno-free cell culture medium. In some embodiments, the culture condition is xeno-free, serum free and feeder-free.

Serum-free and xeno-free medium suitable for culturing the ETV2-induced hematoendothelial progenitor cells to produce non-adherent myeloid progenitors are known in the art and include, but are not limited to, for example, StemLine II (commercially available from Sigma Aldrich).

In some embodiments, a sufficient amount of time for culturing the ETV2-induced HEPs to produce non-adherent myeloid progenitors is at least 4 days, for example, at least 4-23 days. In some embodiments, a sufficient amount of time for culturing the ETV2-induced HEPs to produce non-adherent myeloid progenitors is about 6 to 7 days.

In some embodiments, the methods disclosed herein comprise isolating the non-adherent myeloid cells from the culture. Suitable methods of isolating the cells are known in the art. In one example, non-adherent cells can be collected from the culture leaving the adherent cells behind. In some embodiments, the adherent cells isolated from the non-adherent myeloid cells, may be used in methods of producing macrophages.

For production of macrophages, the methods comprise the step of culturing the myeloid progenitors in a culture medium comprising M-CSF, IL-6, and IL-3 and further culturing the cultured cells comprising M-CSF for a sufficient time to differentiate the myeloid progenitors into modified macrophages. A suitable time to differentiate the myeloid progenitors into modified macrophages includes for at least 9 days, for example at least 9-21 days. In some embodiments, a suitable time for culturing the myeloid progenitors to differentiate into modified macrophages is about 9 to 10 days.

Serum-free and xeno-free medium suitable for culturing the myeloid progenitors to differentiate into modified macrophages are known in the art and include, but are not limited to, for example, StemLine II (commercially available from Sigma Aldrich).

Also provided herein is a population of modified macrophages produced by the methods disclosed herein. The modified macrophages have inhibited expression of SIRPα as well as superior anti-tumor activity for therapeutic purposes. In some embodiments, the modified macrophages also have heightened capacity for antibody-dependent and CAR-dependent cellular phagocytosis without experiencing hypophagia or hypophagia-related exhaustion.

Also provided herein is a population of modified macrophages, wherein the modified macrophages are SIRPA knockout macrophages with a tumor-killing capacity phenotype. In some embodiments, a characteristic of the modified macrophages is a phenotype of heightened capacity for antibody-dependent and CAR-dependent cellular phagocytosis without experiencing hypophagia or macrophage exhaustion. In some embodiments, without being limited by mechanism, a characteristic of the SIRPA knockout macrophages is a phenotype of reduced macrophage exhaustion.

While the methods disclosed herein include ordered, sequential events, the timing of the events may be varied by at least 20%. For example, while a particular step may be disclosed in one embodiment as lasting one day, the event may last for more or less than one day. For example, “one day” may include a period of about 18 to about 30 hours. Periods of time indicated that are multiple day periods may be multiples of “one day,” such as, for example, two days may span a period of about 36 to about 60 hours, and the like. In another embodiment, time variation may be lessened, for example, where day 2 is 48+/−3 hours from d0; day 4 is 96+/−3 hours from d0, and day 5 is 120 hours+/−3 hours from d0.

### Methods for Use of the Modified Macrophages

In particular embodiments, SIRPα knockout macrophages disclosed herein are useful for treating or preventing various disorders such as a cancer. In particular embodiments provided herein is a method of treating cancer comprising administering the SIRPα knockout macrophages disclosed herein. In some embodiments, the method comprises administering the SIRPα-KO macrophages disclosed herein together with a tumor-specific antibody. In some embodiments, the method comprises administering the SIRPα-KO macrophages disclosed herein expressing a chimeric antigen receptor (CAR) that is specific for the tumor target.

The term “chimeric antigen receptor (CAR)” refers to a recombinant fusion protein that has an antigen-specific extracellular domain coupled to an intracellular domain that directs the cell to perform a specialized function upon binding of an antigen to the extracellular domain. In some embodiments, a CAR comprises an antigen-specific extracellular domain (e.g., a single chain variable fragment, scFV, that can bind a surface-expressed antigen of a malignancy, such as GD2) coupled to an intracellular domain (e.g., CD28, CD137, ICOS, CD27, 4-1BB, OX40, CD40L, or CD3z, FcRg) by a transmembrane domain (e.g., derived from a CD4, CD8a, CD28, IgG or CDS-z transmembrane domain).

The antigen-specific extracellular domain of a CAR can recognize and specifically bind an antigen, typically a surface-expressed antigen of a malignancy (e.g., GD2).

An antigen-specific extracellular domain suitable for use in a CAR can be any antigen binding polypeptide, one or more scFv (e.g., anti-GD2 scFv1), or another antibody-based recognition domain (cAb VHH, camelid antibody variable domains) or humanized versions thereof, IgNAR VH (shark antibody variable domains) and humanized versions thereof, sdAb VH (single domain antibody variable domains) and “camelized” antibody variable domains are also suitable for use. In some instances, T cell receptor (TCR)-based recognition domains such as single chain TCR can be used as well as ligands for cytokine receptors.

In certain embodiments, the CAR binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include carbonic anhydrase IX (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-a, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), K-light chain, kinase insert domain receptor (KDR), Lewis Y (LeY), LI cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MARTI, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), and Wilms tumor protein (WT-1), BCMA, NKCS1, EGF1R, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A and ERBB.

The antigen-specific extracellular domain can be linked to the intracellular domain of the CAR by a transmembrane domain, e.g., derived from a CD4, CD8a, CD28, IgG or O\ddot{υ}3-z transmembrane domain. The transmembrane domain traverses the cell membrane, anchors the CAR to the cell surface, and connects the extracellular domain to the intracellular signaling domain, thus impacting expression of the CAR on the cell surface. CARs can also further comprise one or more costimulatory domain and/or one or more spacer. A costimulatory domain can be derived from the intracellular signaling domains of costimulatory proteins that enhance cytokine production, proliferation, cytotoxicity, and/or persistence in vivo. A hinge domain connects (i) the antigen-specific extracellular domain to the transmembrane domain, (ii) the transmembrane domain to a costimulatory domain, (iii) a costimulatory domain to the intracellular domain, and/or (iv) the transmembrane domain to the intracellular domain. For example, inclusion of a hinge domain (e.g., IgGl, IgG2, IgG4, CD28, CD8) between the antigen-specific extracellular domain and the transmembrane domain can affect flexibility of the antigen-binding domain and thereby CAR function. Suitable transmembrane domains, costimulatory domains, and spacers are known in the art.

In one such embodiment the marker is disialoganglioside GD2 (GD2) antigen. Disialoganglioside GD2 (GD2) antigen is highly expressed in a variety of pediatric and adult solid tumors, including neuroblastoma, glioma, and melanoma (Saunder et al., 2017, Expert Review of Anticancer Therapy, 17:889-904). GD2 is usually expressed during fetal development, and its expression in normal post-natal tissues is low, usually limited to osteoprogenitors, the brain, peripheral nerves, and skin melanocytes. Based on these characteristics, a number of GD2-specific immunotherapy strategies have been developed, including GD2-specific antibodies, drug coupling, and chimeric antigen receptor-modified T cell therapy (Richman et al., 2018, Cancer Immunology Research 6:36-46; Louis et al., 2011, Blood 118:6050-6056; Straathof et al., 2020, Sci. Transl. Med. 12).

GD2 is expressed in certain solid tumors, including neuroblastoma, retinoblastoma, medulloblastoma, glioblastoma, melanoma, lung cancer, pancreatic cancer bladder cancer, colorectal cancer, sarcoma, or breast cancer. Neuroblastoma, for example, is a malignancy of the sympathetic nervous system, arising from neural crest progenitors that ordinarily develop into sympathetic ganglia and adrenal medulla. Although heterogeneity in clinical presentation and prognosis is a hallmark of tumor cells that highly and selectively express GD2 antigen, anti-GD2 monoclonal antibodies and GD2-CAR T-cells have been used for targeted immunotherapy. Preclinical data have demonstrated that NK T-cells engineered with a GD2-expressing CAR can target tumor cells directly, and indirectly, by destroying tumor-supporting tumor-associated macrophages (TAMs) in neuroblastoma models (Heczey et al., 2021, American Society of Gene and Cell Therapy Annual Meeting; May 11-14; Virtual. Abstract 19). Melanoma is a type of cancer that develops from the pigment-producing cells known as melanocytes. Many melanoma cells express a range of gangliosides including GD2, GM2, GM3 and GD3 that can be a good choice of target for CAR-mediated therapies (Yvon et al., 2009, Cancer Therapy, 15(18):5852-5860).

This disclosure also provides methods for producing cells that express anti-GD2 CAR. In particular, methods for producing a pluripotent stem cell expressing an anti-GD2 CAR, a CD11b+CD14+ macrophage expressing an anti-GD2 CAR killer cell expressing an anti-GD2 CAR are provided herein. In some embodiments, a nucleic acid vector encoding the chimeric antigen receptor is transfected in human pluripotent stem cells, mesoderm cells, hemangioblasts, hemogenic endothelium cells or hematopoietic progenitor cells for use in any of the methods described herein to produce CAR macrophages. In other embodiments, a nucleic acid vector encoding the anti-GD2 chimeric antigen receptor is transfected in human pluripotent stem cells and then the human pluripotent stem cells can be differentiated to produce a progeny cells (e.g., mesoderm cells, hemangioblast cells, hemogenic endothelium cells, hematopoietic progenitor cells, or macrophages) that also express an anti-GD2 CAR.

The terms “treatment” or “treat,” as used herein, refer to both therapeutic treatment and prophylactic or preventative measures. Those in need of treatment include subjects having cancer as well as those prone to having cancer or those in cancer is to be prevented. In some embodiments, the methods, compositions, and combinations disclosed herein can be used for the treatment of cancer.

In some cases, macrophages obtained according to a method provided herein can be administered as a pharmaceutical composition comprising a therapeutically effective amount of macrophages as a therapeutic agent (i.e., for therapeutic applications).

The terms “pharmaceutical composition” or “therapeutic composition,” as used herein, refer to a compound or composition capable of inducing a desired therapeutic effect when properly administered to a subject. In some embodiments, the disclosure provides a pharmaceutical composition comprising a pharmaceutically acceptable carrier and a therapeutically effective amount of the macrophages of the disclosure.

The terms “pharmaceutically acceptable carrier” or “physiologically acceptable carrier,” as used herein, refer to one or more formulation materials suitable for accomplishing or enhancing the delivery of the macrophages of the disclosure.

The term “subject” is intended to include human and non-human animals, particularly mammals. In certain embodiments, the subject is a human patient.

In exemplary embodiments, CAR macrophages of this disclosure are provided to the subject as a pharmaceutical composition comprising the cells and one or more pharmaceutically acceptable carriers, buffers, or excipients. The pharmaceutical composition for administration must be formulated, produced, and stored according to standard methods that provide proper sterility and stability. Preparations comprising CAR macrophages useful for clinical applications must be obtained in accordance with regulations imposed by governmental agencies such as the U.S. Food and Drug Administration. Accordingly, in exemplary embodiments, the methods provided herein are conducted in accordance with Good Manufacturing Practices (GMPs), Good Tissue Practices (GTPs), and Good Laboratory Practices (GLPs). Reagents comprising animal derived components are not used, and all reagents are purchased from sources that are GMP-compliant. In the context of clinical manufacturing of a cell therapy product, such as in vitro populations of CAR macrophages, GTPs govern donor consent, traceability, and infectious disease screening, whereas the GMP is relevant to the facility, processes, testing, and practices to produce a consistently safe and effective product for human use. See Lu et al., 2009, Stem Cells 27: 2126-2135. Where appropriate, oversight of patient protocols by agencies and institutional panels is envisioned to ensure that informed consent is obtained; safety, bioactivity, appropriate dosage, and efficacy of products are studied in phases; results are statistically significant; and ethical guidelines are followed.

Another aspect of the disclosure provides a method of treating a solid tumor in a subject in need thereof, the method comprising administering a therapeutically effective amount of a genetically engineered CD11b+CD14+ macrophage that expresses anti-GD2 CAR.

Yet another aspect of the disclosure provides a method for reducing the proliferation of a solid tumor cell, the method comprising contacting the solid tumor with a genetically engineered CD11b+CD14+ macrophage that expresses anti-GD2 CAR.

The terms “effective amount” or “therapeutically effective amount” refer to an amount sufficient to effect beneficial or desirable biological and/or clinical results. In other words, a “therapeutically effective” amount is an amount that will provide some alleviation, mitigation, or decrease in at least one clinical symptom in the subject.

The terms “administration” or “administering,” as used herein, refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only and should not be construed as limiting the scope of the disclosure in any way.

### Materials and Method

### Statistical Analysis

Data were analyzed using GraphPad Prism version 9 (GraphPad Software Inc.) and Microsoft Excel (Microsoft Corporation). Tests for statistical significance are listed with each experiment; these included two-sided t-tests for paired analyses and one-way ANOVAs, and two-way ANOVAs for experiments with multiple comparisons of variables or grouped variables, accompanies by the Tukey and Sidak post-hoc test, as inferred to be most appropriate by the software.

### Cancer Cell Culture

SK-OV-3 cells were obtained from American Type Culture Collection (ATCC, Manassas, Virginia, U.S.) and sub-cultured according to ATCC recommendation using McCoy's 5A Medium (ATCC, 30-2007) supplemented with 10% Fetal Bovine Serum (FBS). WM-266-4 cells were obtained by the Department of Dermatology of the University of Wisconsin-Madison and sub-cultured using McCoy's 5A Medium (ATCC, 30-2007) supplemented with 10% FBS. CHLA-136 cells were obtained as a gift from the Children's Hospital of Los Angeles and sub-cultured using Iscove's Modified Dulbecco's Medium (IMDM, Gibco, 12200069) supplemented with 10% FBS.

Human iPSC Maintenance and In Vitro Hematopoietic Differentiation

SIRPA-KO and WT BM9 (IISH2i-BM9-PCBC hiPSC line from WiCell) hiPSCs were maintained and passaged on Cultrex in mTeSR Plus media (WiCell). Hematopoietic differentiation of SIRPA-KO and WT iPSCs was performed on collagen IV (ColIV)-coated plates in IF9S chemically defined serum-free medium as previously described. Anti-GD2-CAR-SIRPA-KO and anti-GD2-CAR hiPSCs were derived from iPS PBMC-3-1 cell line, which was episomally reprogrammed from peripheral blood. Hematopoietic differentiation of anti-GD2-CAR-SIRPA-KO and anti-GD2-CAR iPSCs was performed using a modified arterial endothelium differentiation protocol as described previously.

Generation of iPSC-Macrophages

Floating hematopoietic cells from SIRPA-KO and WT iPSC-derived cell cultures were collected at D9 of differentiation and cultured in IF9S medium with 80 ng/mL M-CSF, 50 ng/mL IL-6, and 10 ng/mL IL-3 on ultra-low attachment plates for 6 days. Fresh media (2 mL/well) was replenished after 3 days. All cells were collected after 6 days and resuspended in IF9S medium supplemented with 80 ng/mL M-CSF on uncoated tissue culture 6-well plates for an additional 4 days. Hematopoietic progenitors from Anti-GD2-CAR-SIRPA-KO and anti-GD2-CAR hiPSCs were induced into iMacs by treatment with E6 media supplemented with 10% FBS, 20 ng/mL M-CSF, 10 ng/mL IL-3, and 20 ng/ml IL-6 for 3 days on uncoated tissue culture plates. All cells were collected and resuspended in fresh E6 media supplemented with 10% FBS and 100 ng/mL M-CSF for an additional 7-15 days. All cytokines were purchased from PeproTech, listed in Table 1.

TABLE 1
Medium Components

| Medium | Day | Day | Day | Day | Day | | Catalog |
|---|---|---|---|---|---|---|---|
| Components | 0-2 | 2-4 | 4-9 | 9-15 | 15-19 | Vendor | # |
| IF9S* | x | x | x | x | x | N/A | N/A |
| FGF2 | x | x | x | | | Peprotech | 100-18B |
| (50 ng/ml) | | | | | | | |
| BMP4 | x | | | | | Peprotech | 120-05 |
| (50 ng/mL) | | | | | | | |
| Activin A | x | | | | | Peprotech | 120-14E |
| (15 ng/ml) | | | | | | | |
| LiCl (2 mM) | x | | | | | Sigma- | 203637- |
| | | | | | | Aldrich | 10G |
| Y-27632 | x | | | | | Tocris | 1254 |
| (10 μM) | | | | | | | |
| VEGF | | x | x | | | Peprotech | 100-20 |
| (50 ng/ml) | | | | | | | |
| SB431542 | | x | | | | Biogems | 3014193 |
| (5 μM) | | | | | | | |
| SCF | | | x | | | Peprotech | 300-07 |
| (50 ng/ml) | | | | | | | |
| TPO | | | x | | | Peprotech | 300-18 |
| (50 ng/mL) | | | | | | | |
| IL-3 | | | x | x | | Peprotech | 200-03 |
| (10 ng/ml) | | | | | | | |
| IL-6 | | | x | x | | Peprotech | 200-06 |
| (50 ng/mL) | | | | | | | |
| M-CSF | | | | x | x | Peprotech | 300-25 |
| (80 ng/ml) | | | | | | | |

*IF9S medium components are previously defined by Uenishi et al., 2014

### CAR Construct and Generation of Anti-GD2 CAR-iPSCs

Anti-GD2 CAR was cloned into AAVS1-DEST vector and integrated into AAVS1 alleles of PBMC-3-1 iPSC line using CRISPR-Cas9 as previously described.

SIRPA-Knockout in hiPSCs

SIRPA gene was targeted for knockout at exon 3 using two sgRNAs, listed in Table 2. Singularized hiPSCs were electroporated with Cas9 protein and both sgRNAs using Lonza Amaxa and Human Stem Cell Nucleofector Starter Kit (Lonza, VPH-5002). After electroporation, cells were serially diluted into 6 well plate containing mTeSR+ media (WiCell) and CloneR supplement (STEMCELL Technologies). After 2 days, CloneR was removed and 5-7 days later, single-cell colonies were selected and expanded for genotyping and further use.

TABLE 2
PCR Primers and sgRNAs

| | Primer | | SEQ |
|---|---|---|---|
| Primers/ | or | | ID |
| sgRNA | sgRNA | Sequence | NO |
| SIRPA | Primer | AATCTTAACACCT | 1 |
| intron 2_F | | TGTACAGCCCCA | |
| SIRPA | Primer | AGTGCCTGCTCCA | 2 |
| exon 3_R | | GACTTAAA | |
| SIRPA | sgRNA | GTGCTCCTTTCCA | 3 |
| sgRNA_1 | | GGAGTGG | |
| SIRPA | sgRNA | ACTTAAACTCCAC | 4 |
| sgRNA_2 | | GTCATCG | |

### Western Blot

For Western Blot experiments, iMacs were generated from hPSCs and harvested for analysis. The cells were lysed using Pierce IP lysis buffer with Pierce protease inhibitors. Protein levels were quantified using the Pierce BCA Assay kit (Thermo Fisher, Waltham, MA) and normalized to 10 ug of total protein prior to running on pre-cast 4-12% gradient SDS-PAGE gels and subsequent transfer to PVDF membranes using Bio-Rad Trans-Blot Turbo System. The membrane was blocked with 5% Difco™ Skim Milk (BD, 232100) in TBST buffer (1%) for human anti-SIRPα antibody (MyBioSource, MBS2026512, 3 ug/mL) and anti-GAPDH (Santa Cruz Biotechnology, 1:5000) for probing. The membranes were incubated with primary antibodies overnight at 4° C. after blocking with mild agitation and were blotted with their corresponding HRP-linked secondary antibodies at room temperature for one hour. Primary and secondary antibodies were diluted in 1% milk TBST buffer. After probing, membranes were washed in 1% TBST buffer for 5 mins three times with mild agitation. Detection of protein on membrane was visualized using Pierce ECL Western Blotting Substrate (Thermo Fisher Scientific Inc., 32209).

### Immunostaining of SIRPα

For immunofluorescence experiments, CAR and CS iMacs were generated from iPSCs and harvested for analysis. iMacs were then fixed in cold methanol for 10 mins at −20° C. and then blocked and permeabilized with 2.5% donkey serum and 0.2% Triton-X100 for 20 mins at room temperature. Human anti-SIRPα (MyBioSource, MBS2026512, 3 ug/mL) was added to the iMacs and incubated for 2 hours at room temperature. Primary antibody was removed, and a secondary anti-Rabbit IgG Alexa Fluor 488 (Invitrogen, A-21206, 2 ug/mL) was applied and incubated for 1 hour at room temperature. In between staining steps, iMacs were washed with PBS. DAPI was used as a nuclear stain control. Images were taken by Nikon confocal microscopy.

### Flow Cytometry

Cells (0.1-1×10⁶) were resuspended in diluted antibodies and incubated at 4° C. for 20-30 minutes. After washing, flow cytometric analysis was performed on MACSQuant Analyzer 10 (Miltenyi Biotech). FlowJo software (FlowJo LLC) was used for the data analysis. Antibodies used in this study are listed in Table 3.

TABLE 3
Antibodies used in this study

| Antibody | Conjugate | Vendor | Catalog # | Application |
|---|---|---|---|---|
| CD43 | FITC | BD Biosciences | 555475 | Flow cytometry |
| CD235a | PE | BD Biosciences | 561051 | Flow cytometry |
| CD41a | PE | BD Biosciences | 555467 | Flow cytometry |
| CD45 | APC | BD Biosciences | 555485 | Flow cytometry |
| CD14 | PE | BD Biosciences | 555398 | Flow cytometry |
| CD163 | BV421 | Biolegend | 333610 | Flow cytometry |
| CD206 | FITC | Biolegend | 321104 | Flow cytometry |
| CD80 | PE-Cy5 | BD Biosciences | 559370 | Flow cytometry |
| CD86 | APC | Miltenyi | 130-116-161 | Flow cytometry |
| HLA-DR | APC | BD Biosciences | 559868 | Flow cytometry |
| CD11b | APC | Miltenyi | 130-091-241 | Flow cytometry |
| CD16 | PE | BD Biosciences | 555407 | Flow cytometry |
| CD18 | APC | BD | 551060 | Flow cytometry |
| CD47 | APC | Biolegend | 323123 | Flow cytometry |
| CD47 | PE | Biolegend | 323108 | Flow cytometry |
| 1A7 | Unconjugated | NCI Repository | NA | Flow cytometry |
| Anti-GD3 (R24) | Unconjugated | NCI Repository | NA | Functional studies |
| Anti-GD2 ( | Unconjugated | NCI Repository | NA | Functional studies |
| Herceptin (anti-HER2) | Unconjugated | Genentech, Inc | NA | Functional studies |
| SIRPa | Unconjugated | MyBioSource | MBS2026512 | Western blot |

### Phagocytosis Assays

iMacs were co-cultured with GFP-expressing cancer cells lines with and without mAb in IF9S media, then harvested and stained with anti-CD45 APC antibody (BD, 555483) for flow cytometric analysis. Cells were then washed with FACS buffer for 1-2 times and flow cytometric analysis was performed on MACSQuant Analyzer 10 (Miltenyi Biotech). Engulfment Index was calculated as (# of DP CD45+GFP+)/(# of total GFP+ cells)×100.

### Cytotoxicity and In Vitro Tumor Burden Assays

iMacs were co-cultured with GFP- and Luciferase-expressing cancer cell lines with or without mAb in respective cancer culture medium. To visualize GFP+ cancer cell viability, co-cultures were subject to fluorescence microscopy (ECHO Revolve). For luciferase-based viability, D-Luciferin substrate was added to co-cultures and bioluminescence was read at 562 nm using SpectraMax plate reader. Untreated cancer-only was used as a spontaneous death control, cancer treated with lysis buffer was used as a maximum cell death control. Relative tumor growth, normalized to 1, was calculated as (experimental−maximum cell death)/(spontaneous death −maximum cell death).

### Macrophage Exhaustion Assay

WT or SIRPA-KO iMacs were co-cultured with SKOV-3 GFP+Luc2+ cells together in IF9S media at two different effector-to-target ratios: 10:1 and 20:1 in the presence of anti-HER2 mAb (1 ug/mL). After 24 hours, cultures were assessed for 1) GFP+ tumor viability by fluorescence microscopy and 2) luciferase activity by adding D-luciferin potassium substrate (VivoGlo, Promega, PAP1041) to the co-culture and reading bioluminescence at 562 nm on a SpectraMax imager. Immediately after, total media of the co-cultures was removed and replenished with the following: 5000 fresh SKOV-3 cancer cells and anti-HER2 (1 ug/mL) in IF9S media. After media replacement, co-cultured were placed back into a normoxic incubator for another 24 hours. This process was repeated four times, for a total of five SKOV-3 and anti-HER2 mAb exposures over the course of 120 hours.

### RNA Sequencing

WT and SIRPA-KO iMacs were either cultured alone for 24 hours, or co-cultured with SK-OV-3 cells (E:T=10:1) and anti-HER2 (1 ug/mL) for 24 hours or 96 hours. All cells were harvested and sorted for CD45+ using anti-human CD45 antibody and anti-CD45 microbeads (Miltenyi, MACS sorter). Total RNA was isolated from the CD45+ sorted populations by using the RNeasy Mini Kit (Qiagen, 74104) and quantified with TapeStation (GENEWIZ by Azenta). The cDNA library was prepared by GENEWIZ by Azenta and ~20 million paired end reads per sample were sequenced on Illumina.

### Bioinformatic Analysis of RNA-Sequencing Data

Bioinformatic analysis of transcriptomic data adhere to recommended ENCODE guidelines and best practices for RNA-Seq. Alignment of adapter-trimmed (Skewer v0.1.123) 2×150 (paired-end; PE) bp strand-specific Illumina reads to the Homo sapiens GRCh38.p10 genome (assembly accession NCBI:GCA_000001405.25) was achieved with the Spliced Transcripts Alignment to a Reference (STAR v2.7.10b) software, a splice-junction aware aligner, using annotation provided by Ensembl. Expression estimation was performed with RSEM v1.3.1 (RNASeq by Expectation Maximization). To test for differential gene expression among individual group contrasts, expected read counts obtained from RSEM were used as input into edgeR (3.42.2) (Dobin, A. et al. (2013) Bioinformatics 29, 15-21). Inter-sample normalization was achieved with the trimmed mean of M-values (TMM) (Li, B. & Dewey, C. N. (2011) BMC Bioinformatics 12, 323). method. Statistical significance of the negative-binomial regression test was adjusted with a Benjamini-Hochberg FDR correction at the 5% level. Prior to statistical analysis with edgeR, independent filtering was applied and required genes to have a count-per-million (CPM) above k in n samples, where k is determined by minimum read count (10 reads) and by the sample library sizes where n is determined by the number of biological replicates in each group. The validity of the Benjamini-Hochberg FDR multiple testing procedure was evaluated by inspection of the uncorrected p-value distribution. Gene set enrichment analysis (GSEA) was performed following criteria outlined by Subramanian et al., 2005.

### Secretome Assay

Secretome assay was performed according to the manufacturer's instruction (U-PLEX assay, Mesoscale Diagnostics).

### In Vivo Mouse Xenograft Experiments

To establish the SK-OV-3 ovarian carcinoma xenograft model, female NSG mice at 6-12 weeks old (The Jackson Laboratory) were injected with 7.5×10⁶ GFP- and luciferase-expressing SK-OV-3 cells via intraperitoneal injection two days prior to treatment. To establish the CHLA-136 neuroblastoma xenograft model, male and female NCG-X mice at 6-12 weeks old (The Jackson Laboratory) were injected with 6×10⁵ via tail vein intravenous injection two days prior to treatment. To assess bioluminescence of tumor burden, mice were anesthetized with isoflurane and injected with D-luciferin and imaged in the IVIS Spectrum In Vivo Imaging System. Images of mice and total flux [photons/s] of tumor bioluminescence was analyzed in Living Image software.

### Example 1: Generation of SIRPA-KO iPSC-Derived Macrophages

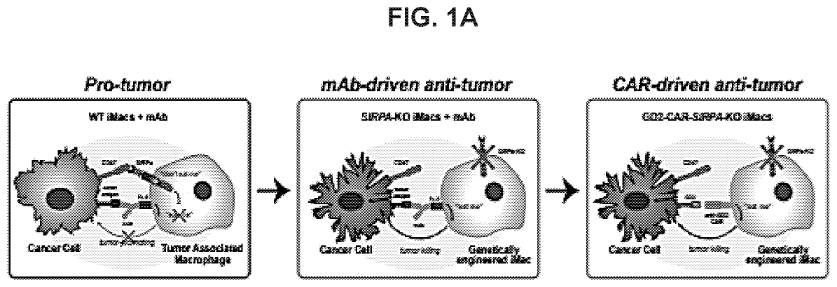

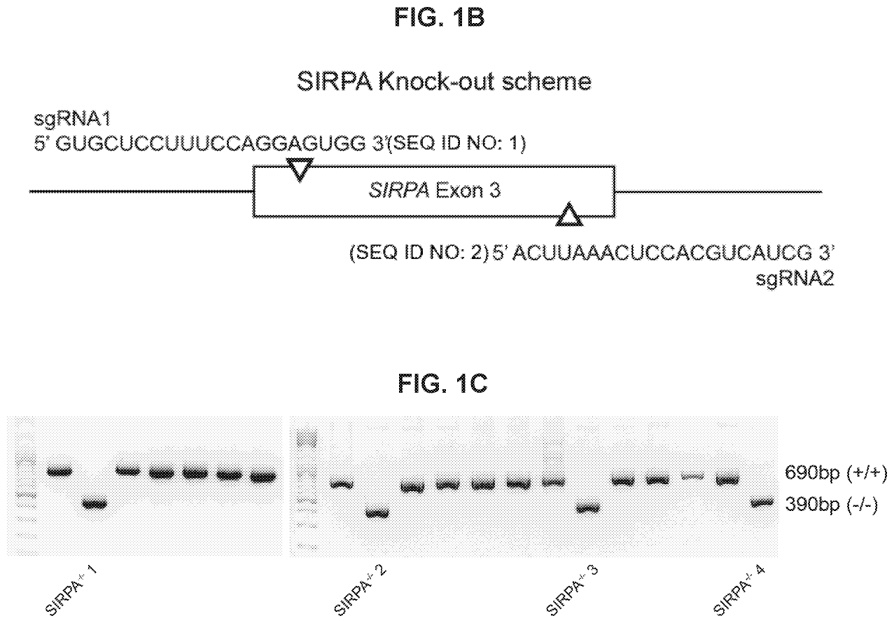

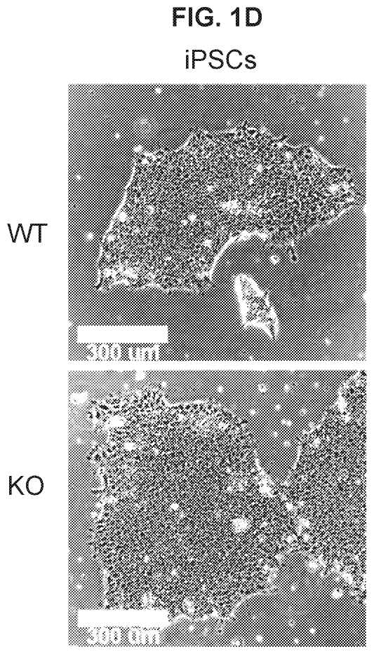

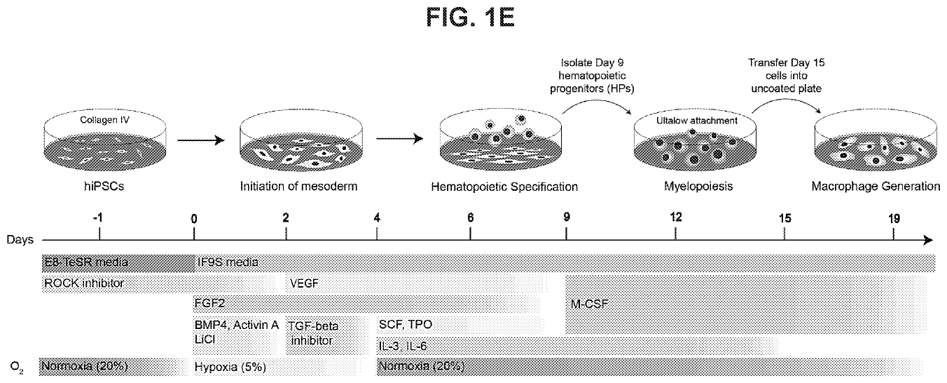

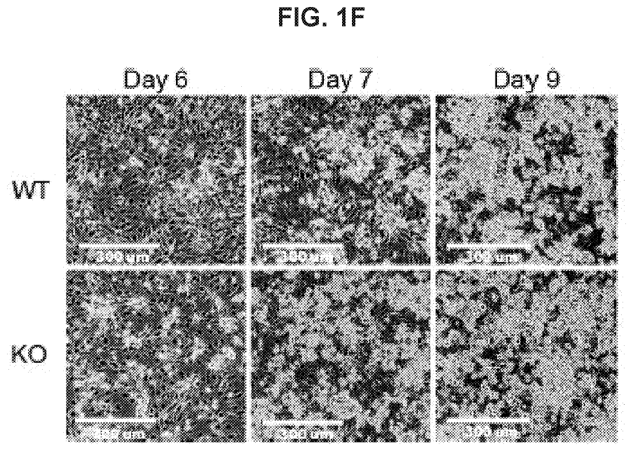

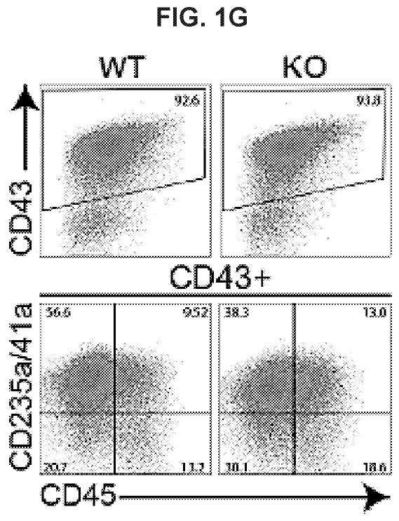

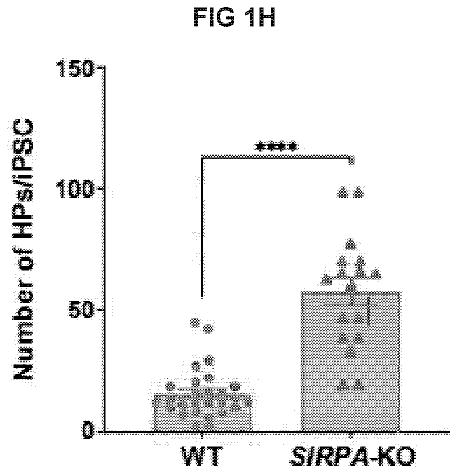

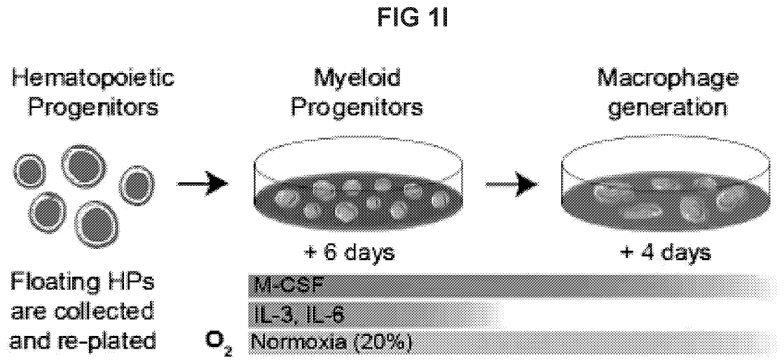

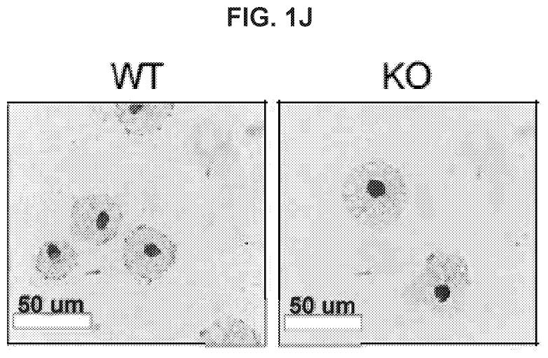

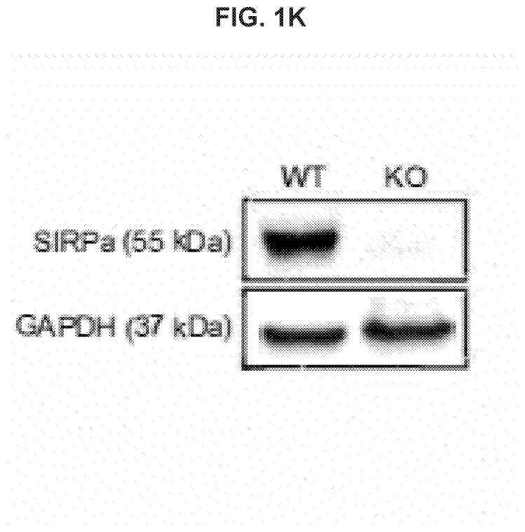

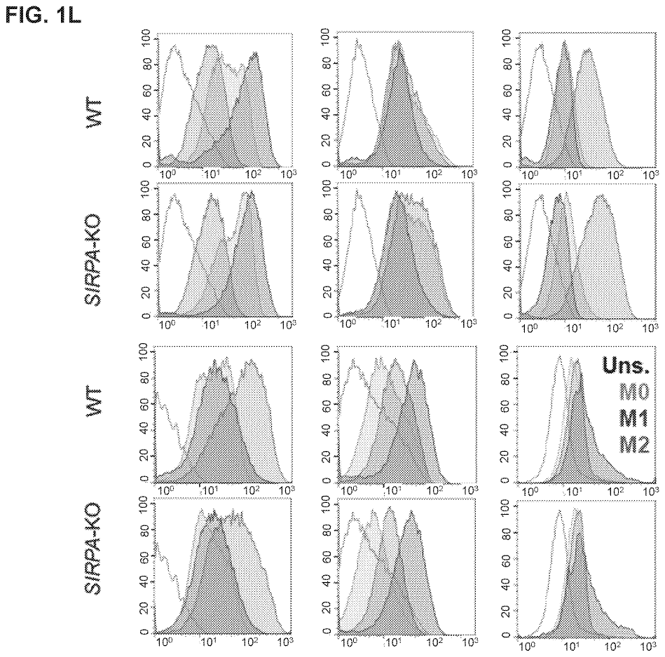

To generate a SIRPA-KO iPSC line, IISH2i-BM9 iPS cells derived from human bone marrow were subjected to CRISPR-Cas9 gene editing via electroporation-based transfection using two synthetic guide RNAs (sgRNAs) flanking exon 3 of SIRPA gene. SIRPA exon 3 was intentionally targeted for deletion due to the multiple CD47-binding motifs within the region (FIG. 1i). After electroporation, single-clone colonies were isolated for genotyping and four SIRPα homozygous knockout clones were selected for further use (FIG. 1C-D).

Previously, a serum-free, xeno-free, component-defined 2D method was developed for in vitro hematopoietic differentiation that utilizes morphogen-driven formation of the hemogenic endothelium by day 5, followed by generation of multipotent hematopoietic progenitors (HPs) with lymphoid and myeloid potential on day 9 (FIG. 1E). Throughout differentiation, SIRPA-KO cells displayed similar morphology to WT, exhibiting the endothelial-to-hematopoietic transition from days 6-9 during in vitro hematopoiesis and produced multipotent HPs expressing CD43, CD45, and CD235a/CD41a markers (FIG. 1F-G). However, the number of HPs derived per one iPSC was significantly higher in SIRPA-KO cell cultures compared to WT, signifying a possible alternative role of SIRPA signaling during blood formation (FIG. 1H).

To generate macrophages, differentiation of HPs with IF9S media was continued and supplemented with myeloid-supportive cytokines M-CSF, IL-3, and IL-6 for 6 days on ultra-low attachment 6-well plates (FIG. 1I). On HP+6 days, SIRPA-KO and WT myeloid progenitor cells displayed a similar heterogenous phenotype with marked expression of CD45, CD14, CD11b, CD16, and CD18, signifying that knocking out SIRPα does not affect the development of myeloid-cell lineage. After HP+6 days, myeloid cell cultures were transferred to uncoated tissue culture plates with IF9S media supplemented with M-CSF for an additional 4 days and resulted in the generation of a near-pure population of macrophages with an appropriate morphology displaying large diameter and vacuolated cytoplasm (FIG. 1I-J). Knockout of SIRPα protein expression was validated by immunoblotting whole cell lysates and immunofluorescence of day 19 WT and SIRPα-KO iMacs using an anti-human SIRPα antibody targeting the extracellular, CD47-binding region of SIRPα receptor (FIG. 1K). To evaluate the plasticity of SIRPα-KO iMacs, day 19 iMacs were treated with M1-promoting LPS+IFN-gamma or M2-promoting IL-4 for 48 hours and analyzed via flow cytometry. Unpolarized (M0), classically polarized (M1), and alternatively polarized (M2) SIRPA-KO iMacs highly resembled the differential expression of common macrophage markers in comparison to WT (FIG. 1L). After iMacs were stimulated with M1-promoting LPS+IFN-gamma, CD14 and M1-markers CD80 and HLA-DR were notably increased by both WT and SIRPA-KO iMacs. However, when stimulated with M2-promoting IL-4, M2-marker CD206 and M1-marker CD86 were increased in both WT and SIRPA-KO iMacs but not WT (FIG. 1L). Overall, the differential expression of macrophage polarization markers in SIRPA-KO iMacs demonstrated SIRPA-KO iMacs' plasticity in response to fluctuations of pro- and anti-inflammatory signaling, an important hallmark of macrophages' immune functions within the TME.

### Example 2: SIRPA-KO iMacs Possess Superior Anti-Body-Dependent Phagocytosis and Cytotoxicity of Solid Tumor Cancer Cells In Vitro

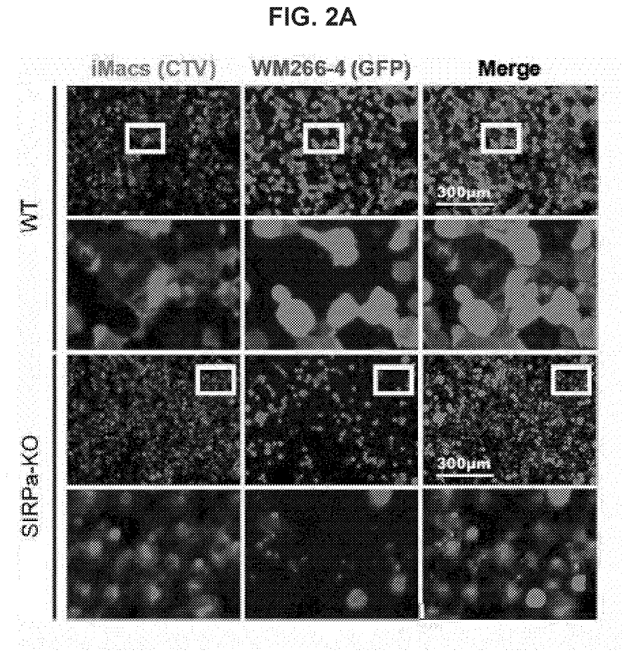

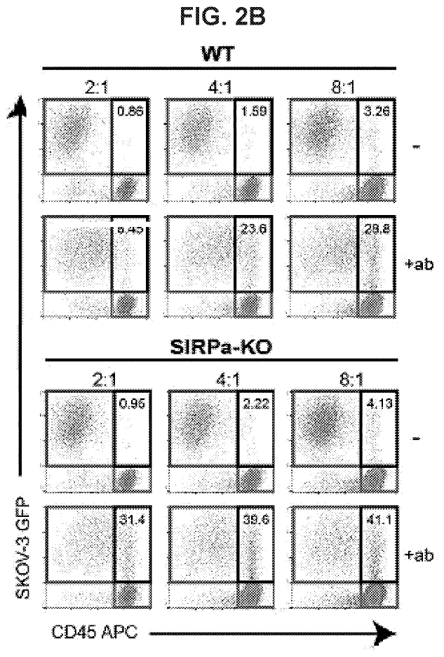

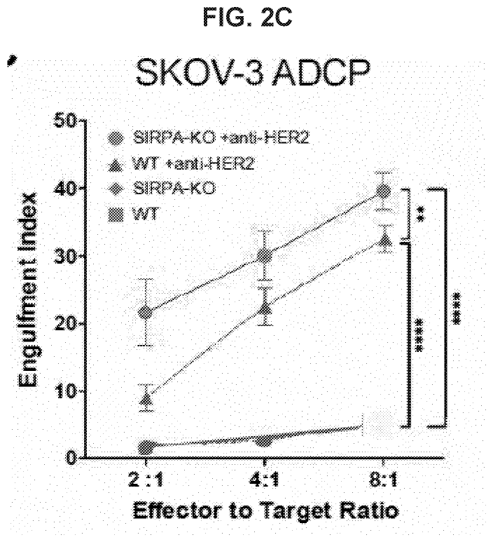

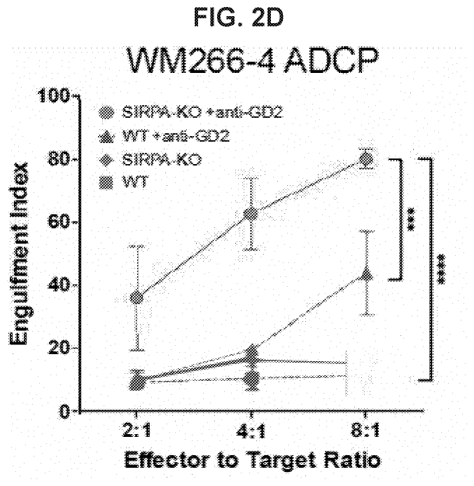

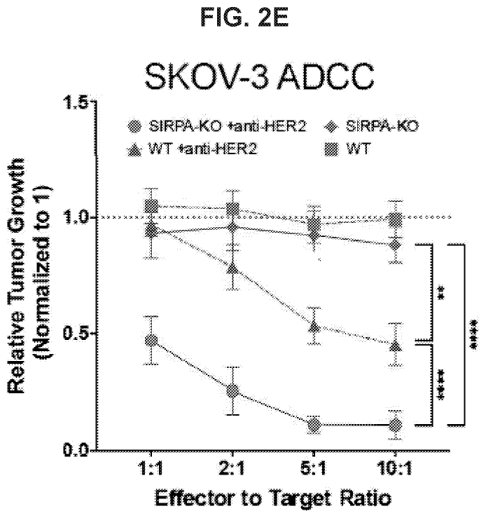

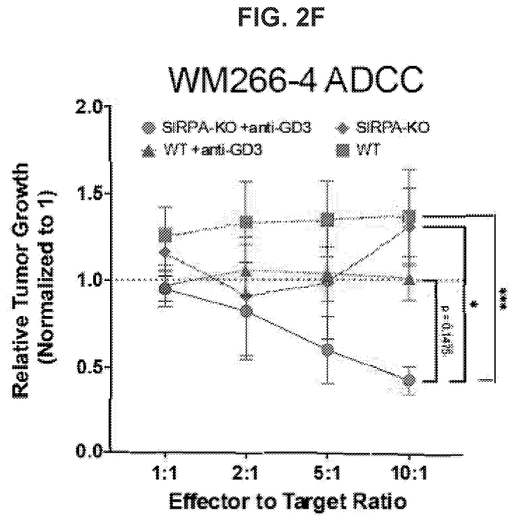

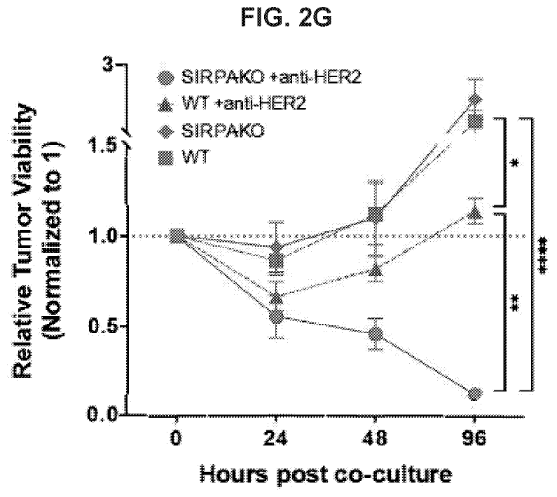

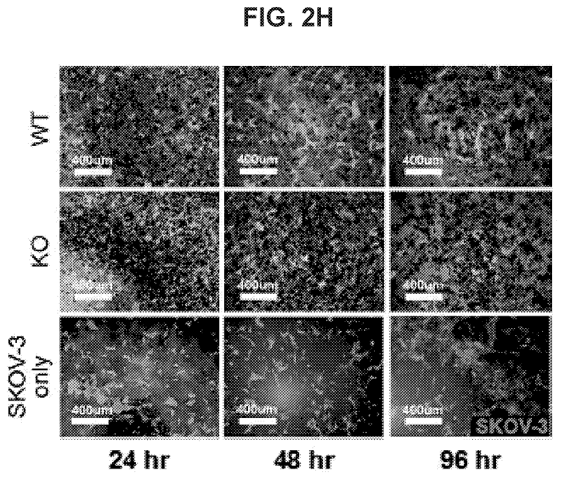

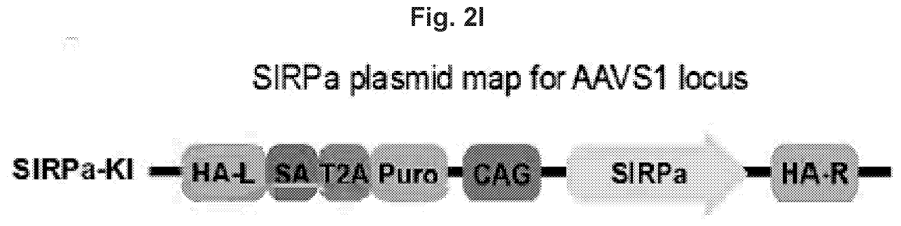

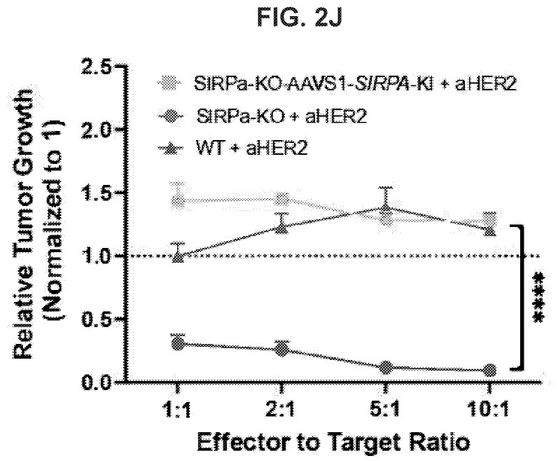

Validation of the model in vitro was conducted using SIRPA-KO iMacs and CD47-expressing cancer cells including SK-OV-3 ovarian cancer, WM-266-4 melanoma, and CHLA-136 neuroblastoma cell lines (FIG. 2A). Co-culturing SIRPA-KO iMacs with SM-266-4 or SK-OV-3 cells without any pro-phagocytic stimuli did not trigger interactions between the iMacs and the cancer cells or produce any anti-tumor response after 5 hours (FIG. 2B-D). Co-culture of SIRPα-KO iMacs with WM-266-4 in the presence of anti-GD3 mAb triggered anti-body dependent cellular phagocytosis (ADCP) as determined by observation of engulfed particles of GFP+WM-266-4 within SIRPA-KO iMacs by immunofluorescent imaging or by flow cytometry (FIG. 2A). The percentages of SIRPA-KO iMacs actively phagocytosing WM-266-4 melanoma, CHLA-163 neuroblastoma and SKOV3 breast carcinoma in the presence of anti-GD3 or anti-HER2 antibodies was increased compared to WT iMacs. (FIG. 2B-D).

iMacs and luciferase-expressing cancer cell lines were co-cultured for 48 in varying effector to target ratios with and without the presence mAb to assess overall tumor-killing capacity of SIRPA-KO iMacs. SIRPA-KO iMacs possessed a heighted capacity for antibody-dependent cellular cytotoxicity (ADCC) against both SK-OV-3 and WM-266-4 cancer cells compared to WT iMacs (FIG. 2E-F).

iMacs and luciferase-expressing cancer cell line were co-cultured for 48 hours in varying effector to target ratios with and without the presence of mAb to assess overall tumor-killing capacity of SIRPA-KO iMacs. SIRPα-KO iMacs showed a heightened capacity for antibody-dependent cellular cytotoxicity (ADCC) against both SK-OV-3 and WM-266-4 cancer cells compared to WT iMacs (FIG. 2E-F). When challenged with SK-OV-3, SIRPα-KO iMacs reached a saturation point at 5:1 effector to target ratio, killing up to 90% target cells, whereas efficacy against WM-266-4 was more limited, capping around 50% cytotoxicity (FIG. 2E-F). Assessment of SIRPα-KO iMacs' antitumor responses in the presence of anti-HER2 mAb over longer durations of time (24, 48, and 96 hours) was conducted since TME can affect macrophage polarization and inhibit anti-tumor activities. SIRPα-KO iMacs stimulated with a single dose of anti-HER2 continually eliminated SK-OV-3 tumor over the entirety of 96 hours, as opposed to WT iMacs that reached peak cytotoxicity at about 30% and were unable to control the outgrowth of SK-OV-3 by 48 hours (FIG. 2G-H). To test that the enhanced anti-tumor activity of iMac was related to SIRPα deletion rather than a potential off-target effect, SIRPα expression was reestablished into the SIRPα-KO iPSC line by inserting SIRPα gene into the AAVS1 locus (FIG. 2I). iMacs generated from the SIRPA-KO-AAVS1-SIRPA-Knock in (KI) iPSCs rescued the WT iMacs' phenotype and were unable to eliminate SK-OV-3 via ADCC (FIG. 2J).

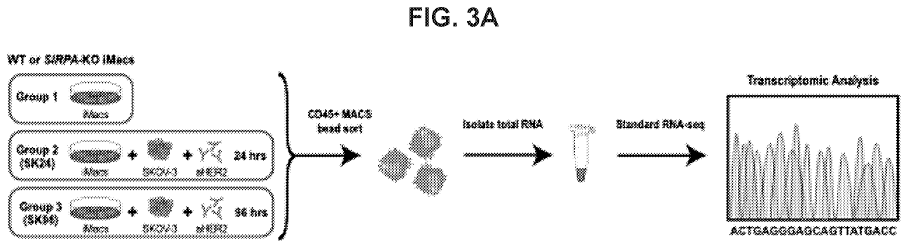

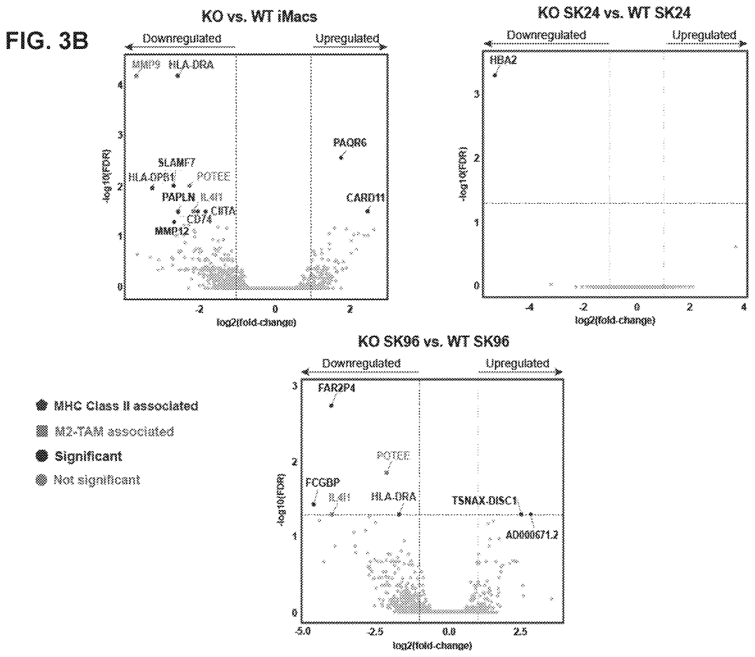

Bulk RNA-sequencing of WT and SIRPA-KO iMacs either cultured alone, with SK-OV-3+anti-HER2 for 4 hours, or with SK-OV-3+anti-HER2 for 96 hours was conducted to assess the transcriptomic differences between WT and SIRPA-KO iMacs before and after tumor challenge (FIG. 3A). Little difference between WT and SIRPA-KO iMacs' transcriptome with or without tumor exposure, despite observing increase in transcripts associated with SKOV3 due to active phagocytosis (FIG. 3B). Of the genes that were differentially expressed between WT and SIRPA-KO iMacs cultured along, a collective downregulation of MHC-class II associated genes by SIRPA-KO iMacs, including HLS-DRA, HLA-DPB1, CD74, and CIITA was observed (FIG. 3B). However, SIRPA-KO iMacs alone that were either M1-, M2-, or un-polarized did not downregulate HLA-DRA protein expression in comparison to WT counterpart, indicating the downregulation of HLA-Class II associated genes may be conserved to the transcriptome and not affect the proteome (FIG. 1K). Additionally, genes associated with pro-tumoral macrophage phenotype including MMP9, IL4I1, and POTEE were significantly downregulated in SIRPA-KO iMacs compared to WT, indicating the possible role SIRPα signaling in macrophages plays in mediating overall macrophage phenotype within the TME (FIG. 3B).

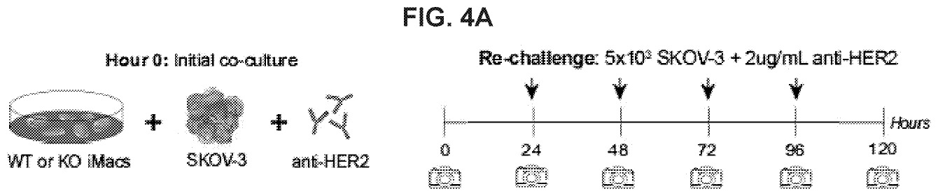

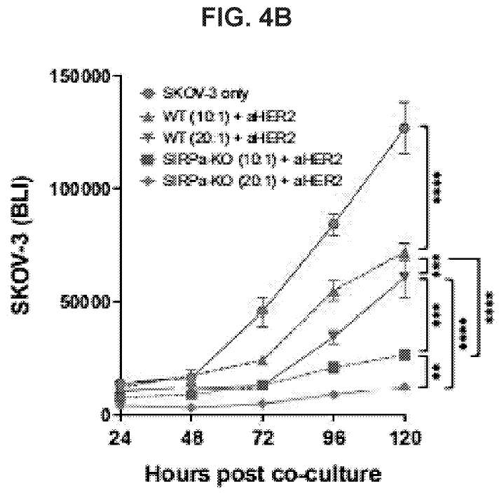

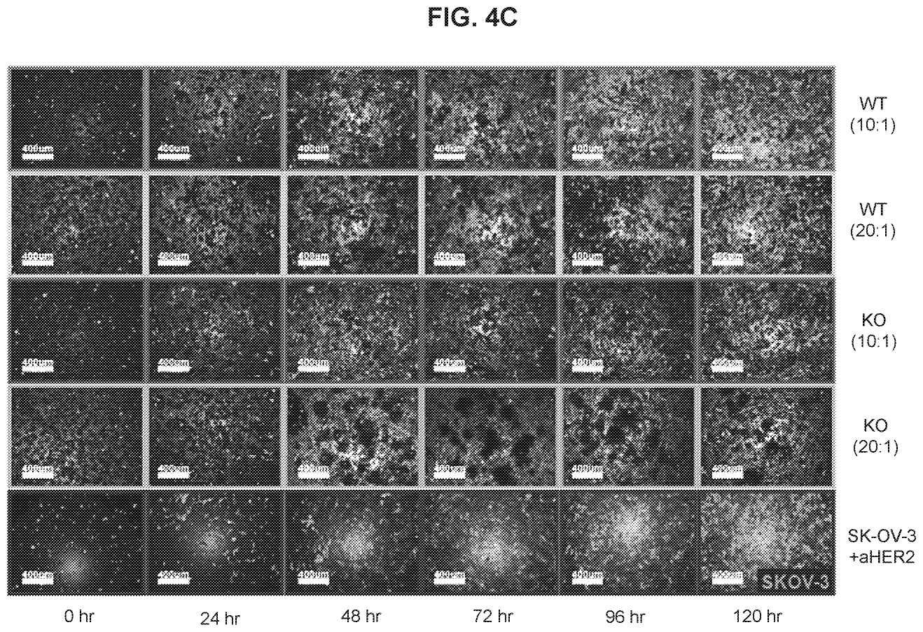

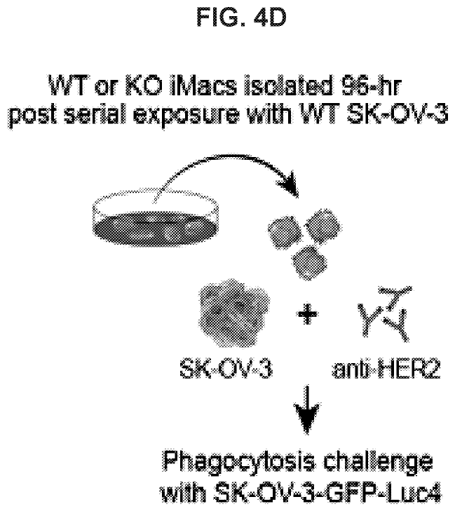

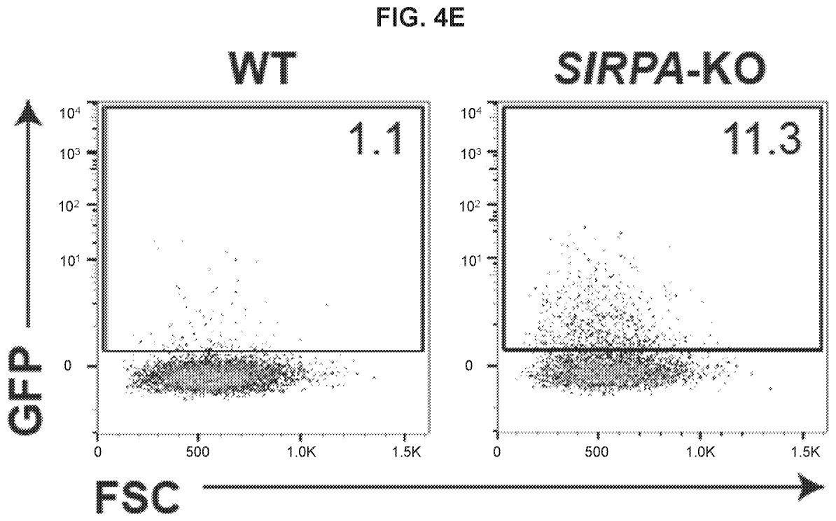

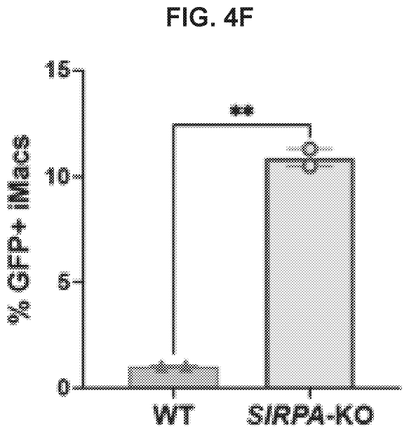

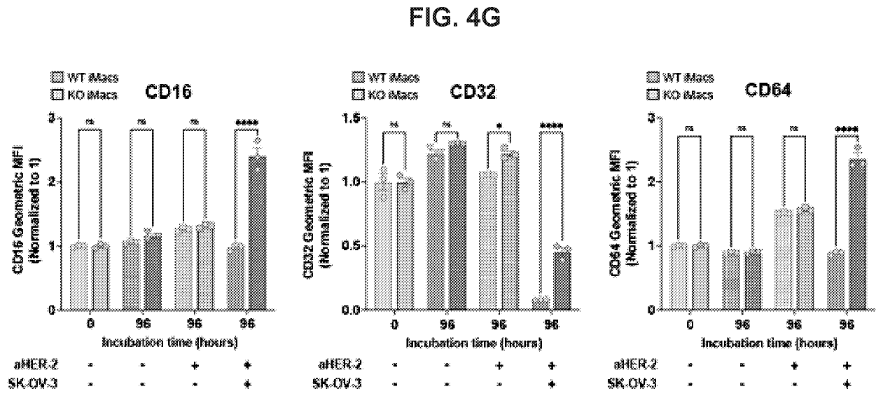

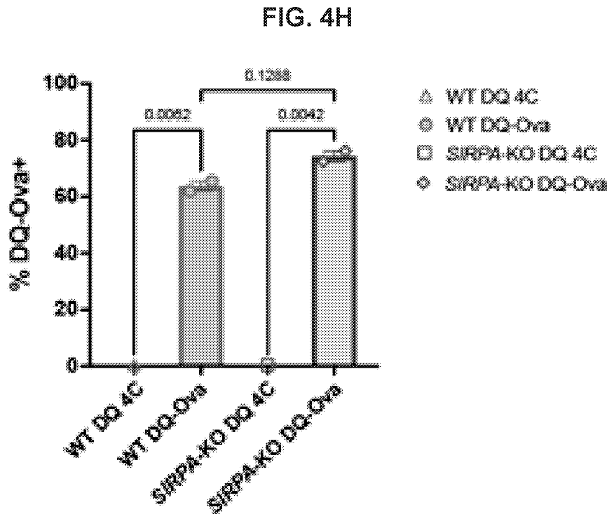

Macrophages exposed to mAb-opsonized target cells display an initial, rapid burst of ADCP, lasting less than an hour, followed by a pronounced decline in phagocytic activity. This pronounced decline results in a refractory period that can persist for days, even with subsequent exposure to mAb-opsonized targets. This diminished capacity of ADCP, referred to as hypophagia or “macrophage exhaustion”, impairs macrophage-mediated clearance of mAb-opsonized target cells and potentially can hinder the antitumor activities against solid tumor cancers. Assessment of cytotoxicity of iMacs following multiple rounds of exposure to SKOV3 GFP-Luc2+ tumor cells in the presence of anti-HER2 antibody was conducted to evaluate the effect of SIRPα ablation on iMac exhaustion. WT or SIRPA-KO iMacs were initially co-cultured with SK-OV-3+anti-HER2 at a 10:1 or 20:1 effector to target ratio at Hour 0. After 24 hours, cultures were assessed for tumor viability either by 1) fluorescence microscopy images of SK-OV-3 for display of GFP viable cancer cells within the co-culture and 2) bioluminescence readings of the co-cultures taken by adding D-luciferin substrate to the co-culture and reading bioluminescence at 562 nm on a spectrophotometer. After these assessments, total media of the co-cultures were removed and replaced with 5,000 additional SK-OV-3 cancer cells+fresh anti-HER2 (1 ug/mL). The co-cultures were re-challenged with fresh SK-OV-3 cancers cells+anti-HER2 every 24 hours until untreated SK-OV-3 cells reached confluency at 120 hours (FIG. 4A-C).

SIRPA-KO iMacs at either 10:1 or 20:1 ratio retained a heightened capacity for ADCC against SK-OV-3 cells for the entirety of the five cancer challenges, while WT iMacs were only able to retain effective antibody-dependent tumor killing up to 72 hours (FIG. 4B-C). Secretomic analysis of iMacs after four re-challenges of SK-OV-3 cancer with and without anti-HER2 were assessed to further evaluate the effect of macrophage exhaustion during cancer re-challenges on SIRPA-KO iMacs. Cytokines associated with pro-inflammatory immunity such as IP-10, IIL-18, IFN-γ, and MIP-la were all diminished in the secretome of SIRPA-KO iMacs+SK-OV-3+anti-HER2 compared to WT counterpart, possibly due to tumor clearance leading to diminished activation within the macrophages. These results indicated that SIRPA-KO iMacs possess superior antibody-dependent antitumor activities in vitro and are resistant to mAb-direct macrophage exhaustion. This allowed SIRPA-KO iMacs to continually engage in antibody-dependent tumor killing after several rounds of cancer re-challenges, unlike the phenotype of WT iMacs.

### Example 3: SIRPA-KO Downregulates HLA Class II Genes and Genes Associated with Pro-Tumoral Phenotype

Bulk RNA sequencing was performed for both WT and SIRPA-KO iMacs either cultured alone, with SK-OV-3+anti-HER2 for 24 hours, or with SK-OV3+anti-HER2 for 96 hours to assess the transcriptomic differences between WT and SIRPA-KO iMacs before and after tumor challenge. While substantia enrichment in transcripts associated with SK-OV-3 was observed due to active phagocytosis after 24 hours, no difference was found between WT and SIRPA-KO iMacs' transcriptome (FIG. 3B-C). However, a small number of genes were found that were differentially expressed between WT and SIRPA-KO iMacs cultured alone, including downregulation of MHC-class II associated genes HLA-DRA, HLA-DPB1, CD74, and CIITA by SIRPA-KO iMacs (FIG. 3B-C). Furthermore, HLA-DRA was observed to also be downregulated after SIRPA-KO iMacs were exposed to SK-OV-3 and anti-HER2 for 96 hours (FIG. 3C). Unstimulated SIRPA-KO iMacs or iMacs exposed to SK-OV-3 and anti-HER2 for 96 hours downregulated MMP9 (Liu et al. (2019) Biomedicine & Pharmacotherapy 117, 109096; Pelekanou et al. (2018) Breast Cancer Research 20, 154; Xu et al. (2019) Amer. J. Trans. Res. 11 6040), IL4I1 (Yue et al. (2015) PLoS ONE 10: e0142979; Sadik et al. (2020) Cell 182: 1252-1270.e34), and POTEE (Vekariya et al. (2019) Cell Immunol 335: 30-40) genes associated with pro-tumoral macrophage phenotype, indicating that the active SIRPα signaling may favor macrophage polarization toward anti-inflammatory M2-like phenotype within the TME (FIG. 3C).

### Example 4: SIRPα Ablation in iMacs Reverse mAb-Mediated Hypophonia-Related Exhaustion

Macrophages exposed to mAb-opsonized target cells display an initial, rapid burst of ADCP, lasting less than an hour, followed by a decline in phagocytic activity persisting for days, even with subsequent exposure to mAb-opsonized targets (Pinney et al. (2020) Blood 136(18):2065-79). This markedly diminished capacity of ADCP, also known as hypophagia, impairs macrophage-mediated clearance of mAb-opsonized target cells, and can be considered a form of macrophage exhaustion that hinders antitumor activity. Cytotoxicity of iMacs was assessed after multiple rounds of exposure to SK-OV-3 GFP-Lac2+ tumor cells in the presence of anti-HER2 antibody to evaluate the effect of SIRPα ablation on iMac exhaustion. WT or SIRPA-KO iMacs were initially co-cultured with SK-OV-3+anti-HER2 at a 10:1 or 20:1 effector to target ratio and re-challenged with fresh SK-OV-3 cancer cells+anti-HER2 every 24 hours until untreated SK-OV-3 cells reached confluency at 120 hours (FIG. 4A-C). SIRPA-KO iMacs were found to retain a heightened capacity for ADCC against SK-OV-3 cells for the entirety of the five cancer challenges, unlike WT iMacs which were only able to retain effective antibody-dependent tumor killing up to 72 hours (FIG. 4B-C). Addition of anti-HER2 alone to SK-OV-3 did not control tumor growth, indicating tumor-killing was due to the presence of SIRPA-KO iMacs and anti-HER2 together (FIG. 4B-C). To validate that the ablation of SIRPα is truly preventing hypophagia, the chronic pronounced decline of ADCP in macrophages, WT and SIRPA-KO iMacs were isolated after 96 hours of serial exposure with unmodified SK-OV-3 and re-challenged with fresh SK-OV-3 expressing GFP and anti-HER2 for a 2-hour phagocytosis assay (FIG. 4D). As anticipated, SIRPA-KO iMacs possessed a considerably heightened capacity for ADCP, and engulfed nearly 10-fold the number of GFP+ tumor targets compared to WT iMacs (FIG. 4E-F).

Previous studies with lymphoma cells had demonstrated that loss and proteolytic degradation of activating Fc receptors are primary causes of hypophagia (Pinney et al. (2020)). Comparison of expression of FcRI (CD64), FcRII (CD32), and FcRIII (CD16) at post 96 hours post-serial tumor exposure demonstrated their downregulation, with CD32 showing the more pronounced decrease. In contrast, SIRPA-KO iMacs upregulated CD16 and CD64 and demonstrated better retention of CD32 expression (FIG. 4G). Additionally, rapid downregulation of CD64 and CD16 were confirmed after short exposure of iMacs to SK-OV-3 and mAbs, with heightened Fc receptor expression in SIRPA-KO iMacs compared to WT cells. No differences in iMac viability were observed between the groups. The defect in phagocytic activity was tumor-specific and did not affect the overall antigen-processing ability of WT and SIRPA-KO iMacs after serial tumor exposure, as shown by the DQ-Ova assay (FIG. 4H).

To determine whether resistance to exhaustion in SIRPA-KO iMacs could be attributed to cytokines, secretome analysis of iMacs of iMacs was performed for four re-challenges of SK-OV-3 cancer with and without anti-HER2. These studies revealed that cytokines associated with pro-inflammatory immunity such as IP-10, IL-18, IFN-γ, and MIP-la were all diminished to the secretome of SIRPA-KO iMacs+SK-OV-3+anti-HER2 compared to WT counterpart (FIG. 4I) likely due to tumor clearance and reduced activation within iMacs. Cumulatively, these results indicate that SIRPA-KO iMacs possess superior Ab-dependent antitumor activities in vitro and are resistant to mAb-directed macrophage exhaustion. This phenomenon allowed SIRPA-KO iMacs to continually engage in Ab-dependent tumor killing after several rounds of cancer re-challenges, unlike the phenotype of WT iMacs.

These data indicated that SIRPA-KO iMacs not only possess superior antitumor activity due to the interruption in the “don't eat me” CD47-SIRPα pathway, but that SIRPα serves as a key regulator of hypophagia and macrophage-exhaustion in the context of chronic tumor and mAb exposure. Furthermore, the ablation of SIRPα in macrophages reversed hypophagia-related exhaustion and promoted an “everlasting” tumor-killing capacity phenotype that WT iMacs do not possess.

### Example 5: SIRPA-KO iMacs Administered with HER2 Antibody Improves Survival of Mice with SKOV3 Xenograft

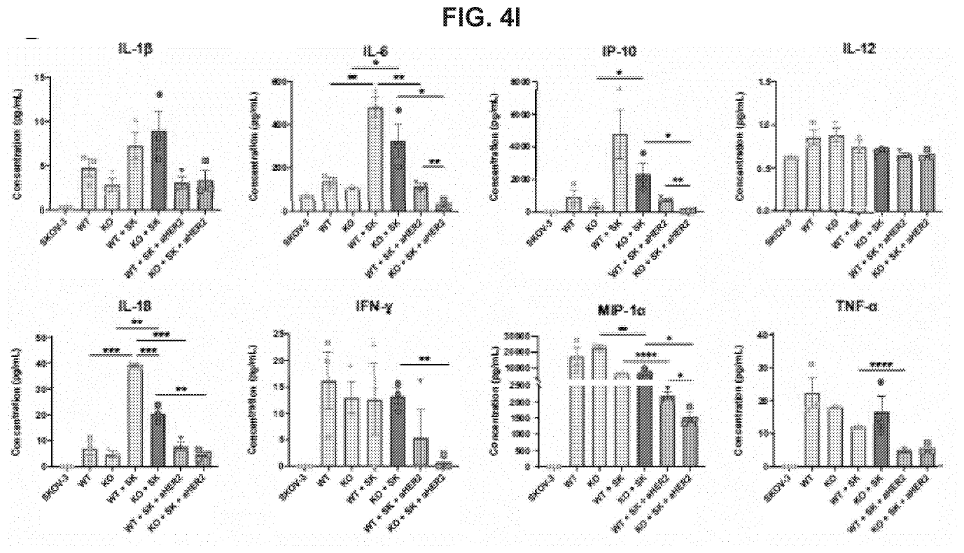

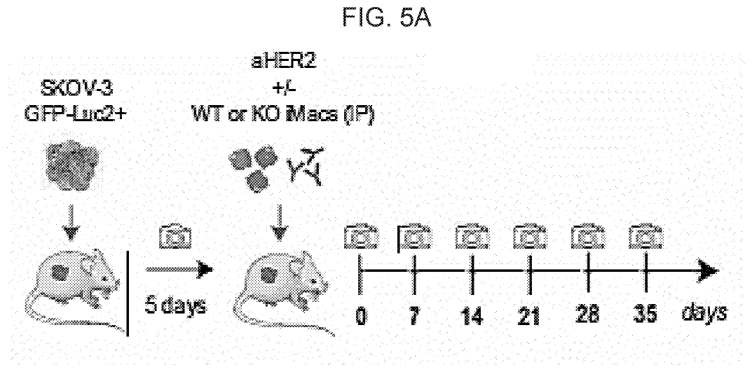

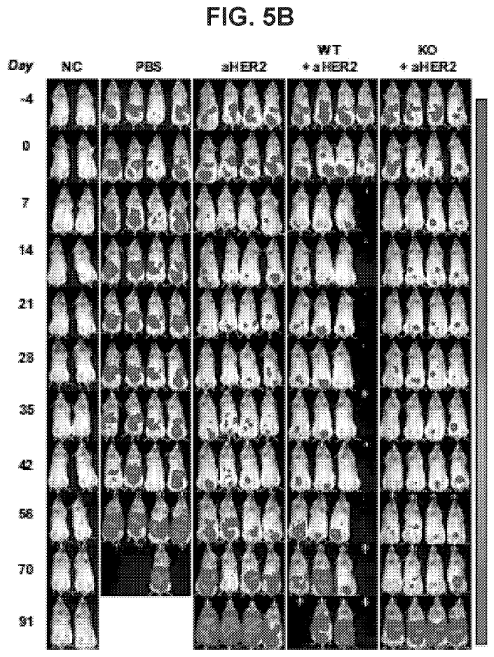

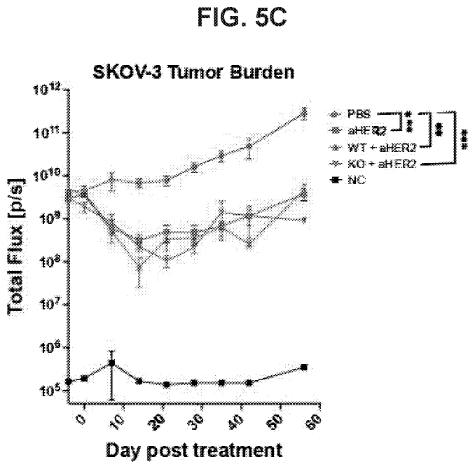

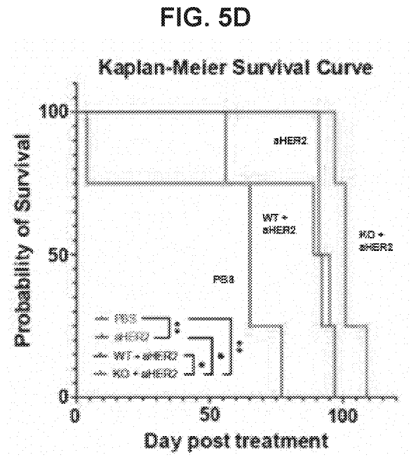

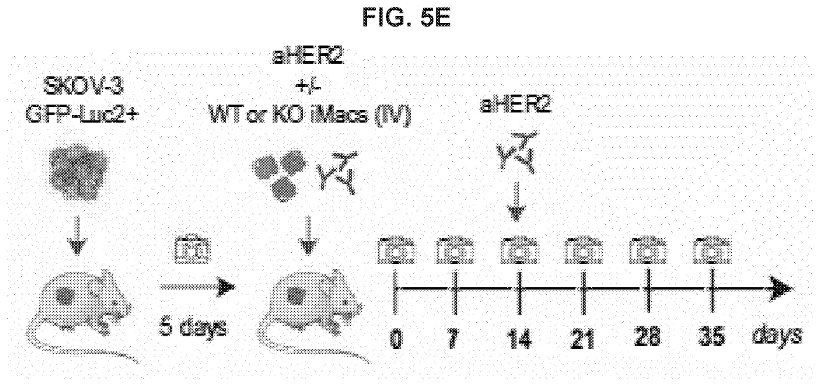

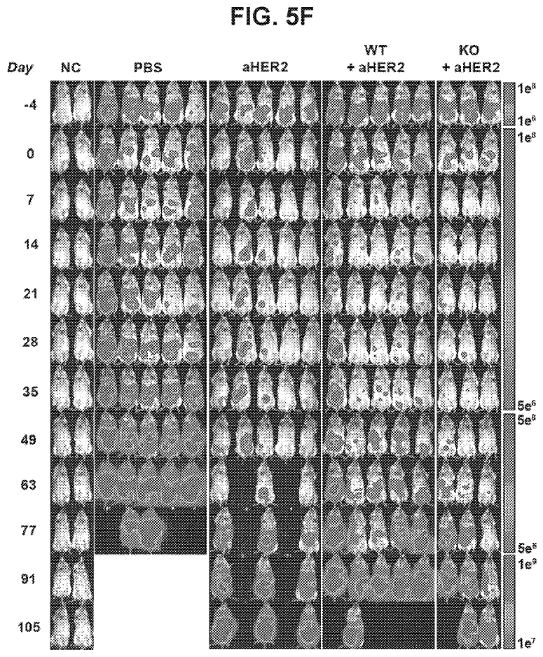

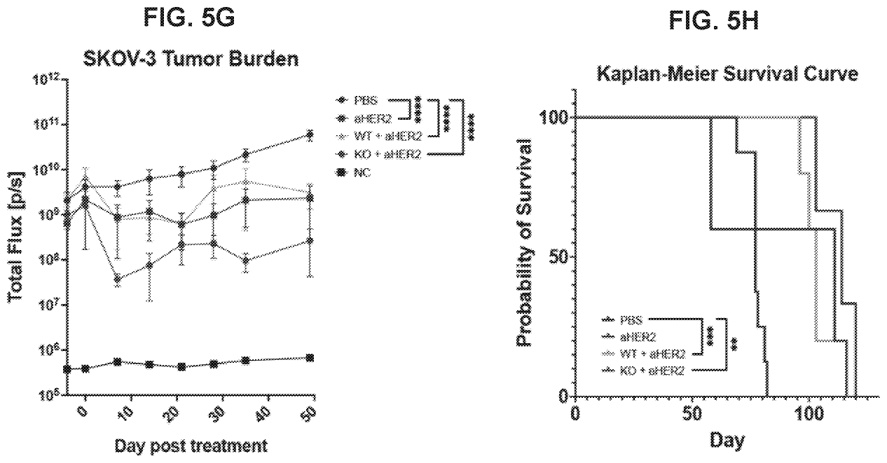

SIRPA-KO iMacs efficiency in vivo was tested by engrafting NSG female mice with human SK-OV-3 ovarian cancer using an intraperitoneal (IP) injection to mimic ovarian cancer progression through the abdomen and peritoneal cavity that is often associated with later stages of ovarian cancer (FIG. 5A, E). No differences in SK-OV-3 tumor burden reduction amongst anti-HER2, WT iMacs+anti-HER2, and SIRPA-KO iMacs+anti-HER2 treatment groups was observed when a single dose of iMacs and mAb was administered via IP injections five days after engraftment (FIG. 5B-C). However, treatment with SIRPA-KO iMacs+anti-HER2 provided a longer survival time than the other treatment groups, suggesting a potential difference in the anti-solid tumor activities of SIRPA-KO iMac in comparison to WT iMacs (FIG. 5D).

Mice were treated five days after SK-OV-3 engraftment with either anti-HER2 alone, WT iMacs+anti-HER2, or SIRPA-KO iMacs+anti-HER2, followed by a second dose of anti-HER2 two weeks after the initial treatment in order to assess whether an antitumor effect can be improved by injecting iMacs intravenously (IV) (FIG. 5E). The SIRPA-KO iMacs administered via IV+anti-HER2 treatment immediately reduced tumor burden within one weeks compared to other treatment groups and continued to suppress tumor growth for 60 days (FIG. 5F-G). The IV injection of SIRPA-KO+anti-HER2 resulted in the longest median survival time for SK-OV-3 engrafted NSG mice, supporting their efficacy in reducing tumor (FIG. 5H). These experiments demonstrated that combination therapy using SIRPA-KO iMacs+anti-HER2 reduced ovarian cancer tumor burden in vivo.

### Example 6: Ablating SIRPα in CAR-iMacs Markedly Increased their Anti-Tumor Activities

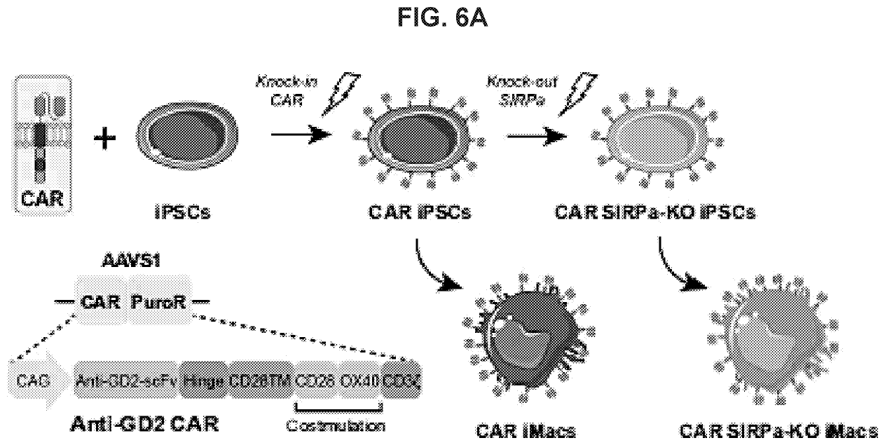

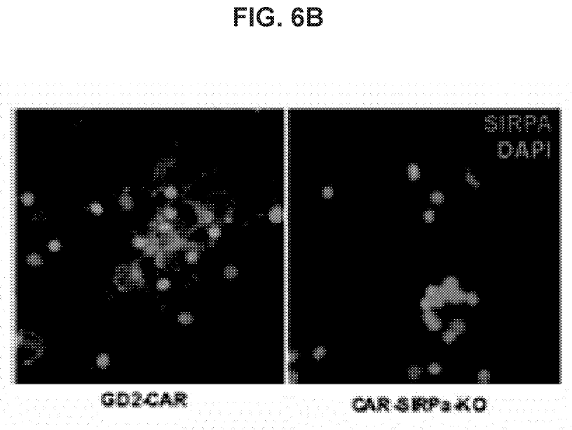

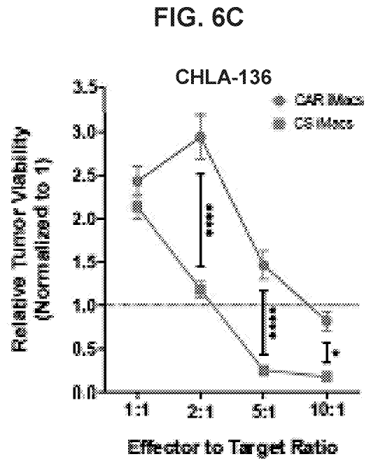

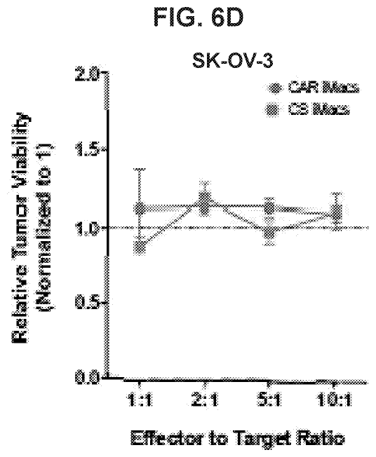

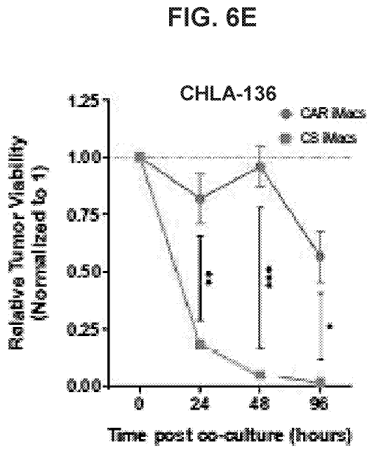

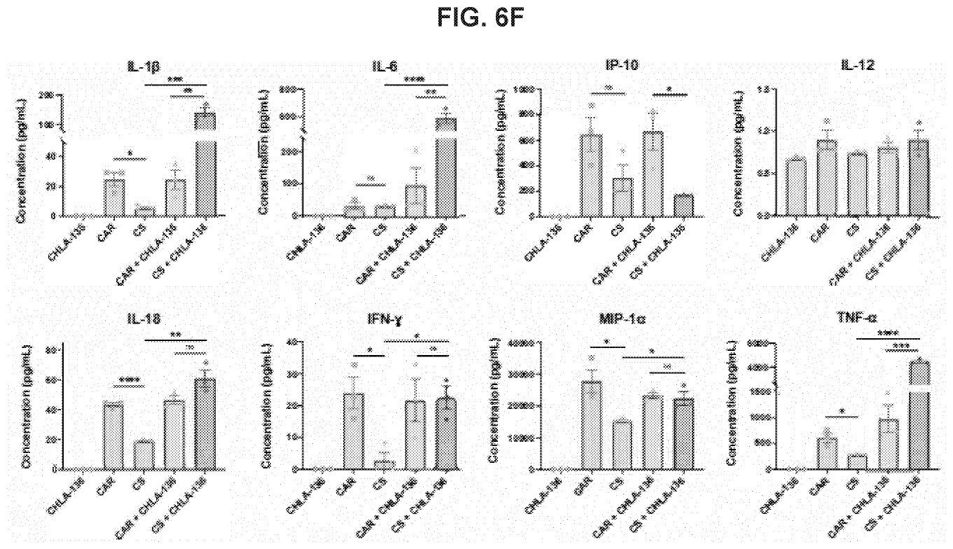

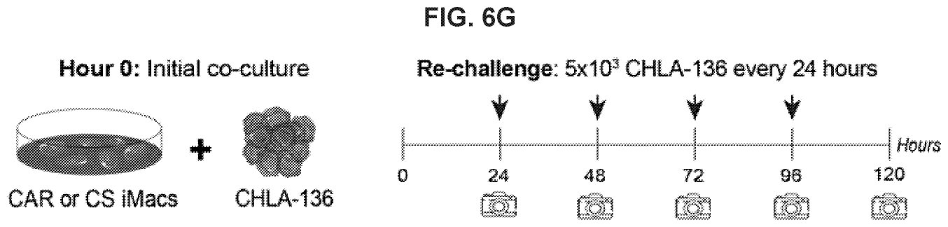

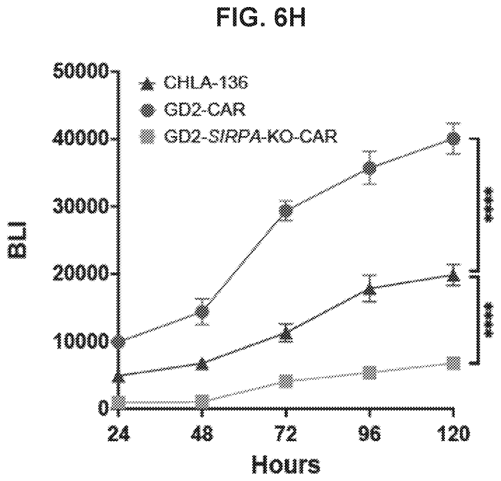

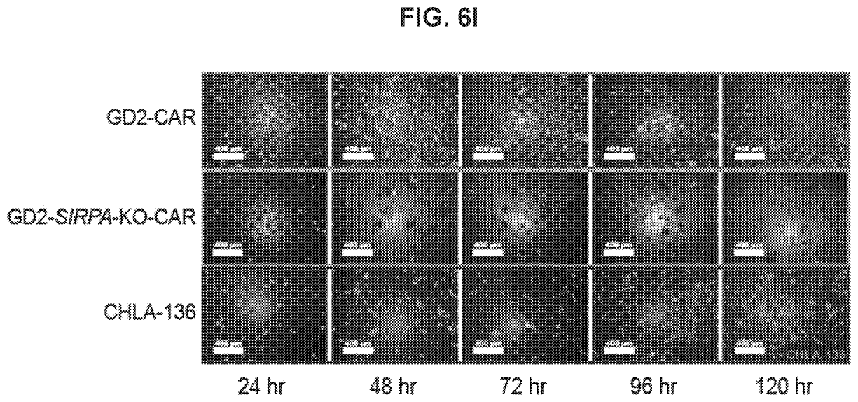

Given the limited antibody-directed effect of SIRPA-KO iMacs on tumor growth in vivo, an assessment of whether knocking out SIRPα would bolster the tumor-killing capacity of anti-disialoganglioside GD2 chimeric antigen receptor (CAR)-expressing iMacs against CD47 solid tumors was conducted. The disialoganglioside (GD2) antigen is highly expressed in a variety of pediatric and adult tumors, including melanoma, neuroblastoma, high grade glioma, osteosarcoma, triple negative breast cancer and non-small cell lung carcinoma, while its expression in normal post-natal tissues is low and limited to peripheral nerve pain fibers. Following SIRPα knock out in human iPSC expressing third-generation anti-GD2 CAR, as described herein (FIG. 6A-B), assessment of antitumor cytotoxicity of CAR-SIRPA-KO iMacs (CS-iMacs) against GD2-expressing neuroblastoma CHLA-136 GFP-Luc2+ and GD2-negative ovarian carcinoma SKOV3 was carried out. CS-iMacs possessed significantly heightened antitumor cytotoxicity against CHLA-136 neuroblastoma compared to CAR-iMacs while both failed to kill SK-OV-3 tumor at any ratio, indicating the specificity to CD2-expressing target cells (FIG. 6C-D).

Evaluation of CS-iMacs' capacity to sustain antitumor activities over longer spans of time during a challenge with GD2 expressing neuroblastoma and melanoma for 96 hours was conducted in light of TME's ability to polarize macrophages to a pro-tumoral phenotype and inhibit their antitumor functions. CS-iMacs possessed an increased initial cytotoxicity against CHLA-136 compared to CAR-iMacs, then continually inhibited tumor growth for 96 hours until nearly 99% of the tumor burden is eliminated (FIG. 6E). Additionally, CS-iMacs reduced WM-266-4 melanoma by 96 hours compared to CAR-iMacs, signifying the ablation of SIRPα significantly heights antitumor efficacy of CAR-iMacs against multiple CD2-expressing solid tumor cell lines. Evaluation of the secretome of CS-iMacs demonstrated the IL-B, IL-6, and TNF-α pro-inflammatory cytokine secretion was more pronounced in CS-iMac cultures with tumor than CAR-iMacs (FIG. 6F) implying that the initial increase of tumor-killing observed from CS-iMacs could be correlated with the activation of pro-inflammatory signaling pathways (FIG. 6F).

Because SIRPα ablation protected macrophages from mAb-driven exhaustion (as shown in FIG. 4A-I), investigation into whether re-exposing CAR-iMacs to multiple rounds of tumor induced their exhaustion, and whether SIRPα ablation effected this process, was pursued. To do so, GD2-CAR or GD2-SIRPA-KO-CAR-iMacs were initially co-cultured at a 10:1 effector-to-target ratio with CD47-expressing CHLA-136 neuroblastoma for 24 hours and were re-exposed 5 times to additional GHLA-136 cells every 24 hours (FIG. 6G). GD2-CAR-iMacs failed to clear tumor cells within 24 hours and promoted tumor growth throughout the remainder of the tumor exposures (FIG. 6H-I), consistent with previous findings that GD2-CAR-iMacs necessitated the entire 96 hours to kill half the GHLS-136 cells during only a single tumor exposure (FIG. 6E). Conversely, GD-SIRPA-KO-CAR-iMacs efficiently killed approximately 80% of the GHLA-136 tumor upon initial exposure at 24 hours and continually sequestered tumor growth for the entire five tumor exposures (FIG. 6H-I).

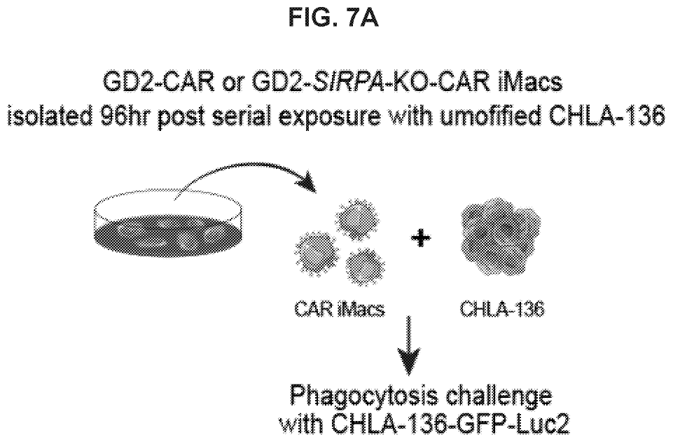

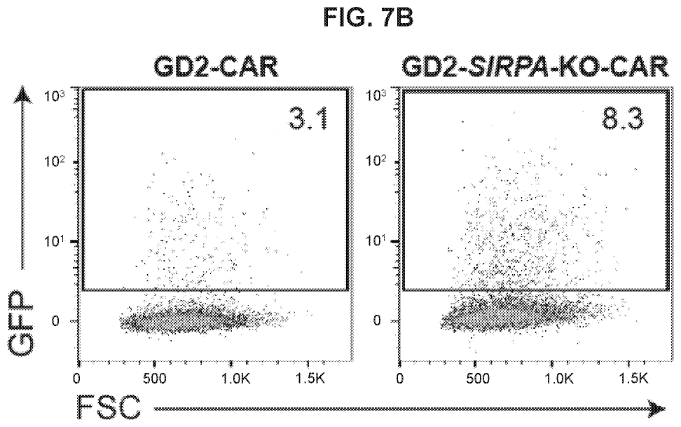

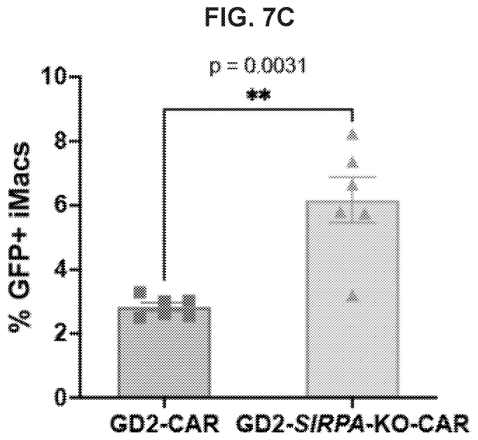

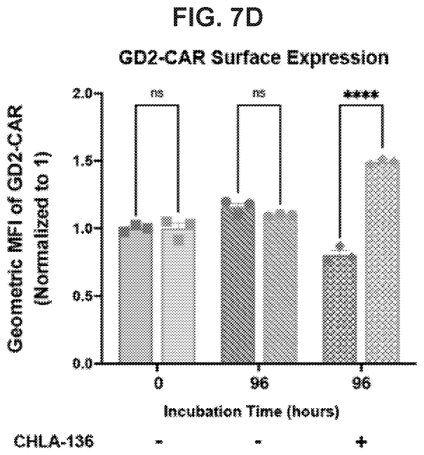

CAR iMacs were serially exposed to unmodified CHLA-136 cells for 96 hours to confirm the heightened capacity for phagocytosis of GFP-Luc2 tumor targets by GD2-SIRPA-KO-CAR-iMacs compared to SIRPA-intact GD2-CAR iMacs (FIG. 7A-C). In view of the impact of SIRPα on surface Fc receptor expression, further investigation was conducted into whether the improved tumor-killing in SIRPA-ablated GD2-CAR iMacs was caused by better retention of GD2-CAR expression. Indeed, after 96 hours of serial tumor exposure, GD2-CAR iMacs had reduced expression of GD2-CAR, whereas GD2-SIRPA-KO-CAR-iMacs possessed significantly heightened GD2-CAR expression in comparison to SIRPα-intact GD2-CAR iMacs (FIG. 7D). These results indicated that SIRPA ablation protected CAR-expressing iMacs from loss of surface CAR expression and CAR-directed hypophagia against tumor targets, allowing for heightened phagocytic capacity even after serial tumor exposure.

### Example 7: SIRPα Ablation in GD2 CAR-iMacs Reduces Metastatic Neuroblastoma Burden In Vivo

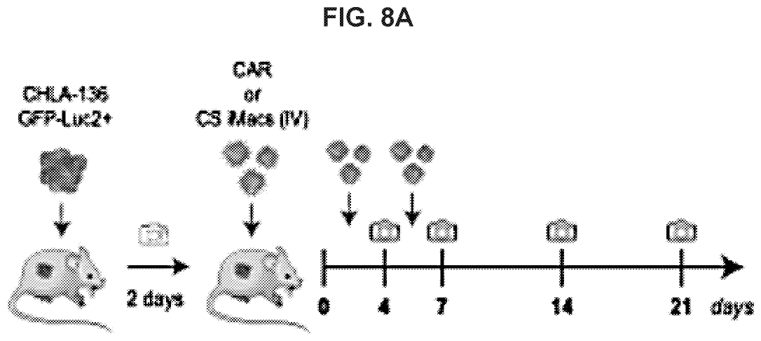

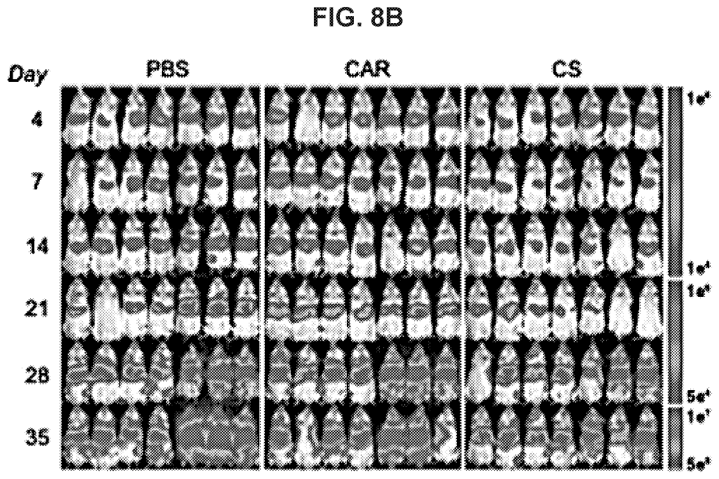

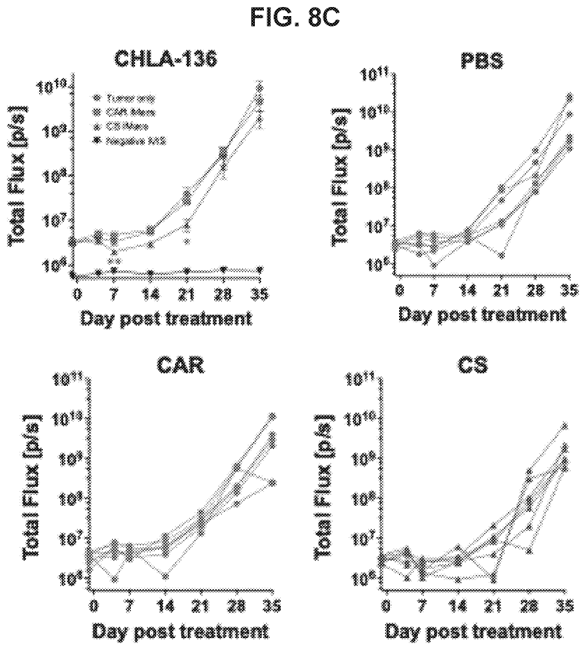

NCG-X mice were engrafted with human CHLA-136 neuroblastoma through intravenous (IV) injection, simulating advanced-stage metastatic neuroblastoma, to evaluate the efficiency of CS-Ms in an in vivo cancer model (FIG. 8A). This xenograft model was chosen due to the highly metastatic nature of neuroblastoma, as relapse or progression of metastatic sites are a major cause of mortality in neuroblastoma pediatric patients (London, W. B. et al. (2017) Cancer 123, 4914-4923; Liu, S. et al. (2023) World J Surg Oncol 21, 130). CS-iMacs were found to markedly delay tumor burden one week after initial administration of therapy and continually inhibit tumor growth significantly more than CAR-iMacs by day 21 (FIG. 8B-C). However, a significant impact of treatment was not observed for mice survival. Overall, ablating SIRPA within CAR macrophages significantly improved their CAR-dependent antitumor capacity in vitro against CD2-expressing solid tumor cancers, enhanced pro-inflammatory cytokine expression, and mitigated neuroblastoma tumor burden xenograft in vivo.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

1-2. (canceled)

3. A method of treating a subject bearing a SIRPα ligand-expressing tumor, or reducing the growth of signal regulatory protein alpha (SIRPα) ligand-expressing tumor cells, comprising contacting the tumor with an effective amount of a combination of SIRPA knockout macrophages, wherein the SIRPA knockout macrophages:
(a) are administered in combination with an antibody immunologically specific for the tumor cells; or
(b) express a chimeric antigen receptor (CAR) that is immunologically specific for the tumor cells:
wherein the tumor cell reducing or eliminating activity of the SIRPA-KO macrophages is characterized by reduced macrophage exhaustion thereby.

4. (canceled)

5. The methods of claim 3 wherein SIRPα ligand expressed by the tumor cells is CD47.

6. The methods of claim 3 wherein modified macrophages are produced from pluripotent stem cells by a method comprising:
(a) culturing human pluripotent stem cells having inhibited expression of SIRPα in normoxic conditions for about 24 hours in serum-free culture medium comprising of L-ascorbic acid-2-phosphate magnesium, sodium selenium, transferrin, insulin, NaHCO₃, fibroblast growth factor 2 (FGF2), transforming growth factor beta 1 (TGFβ1), and a Rho kinase (ROCK) inhibitor;
(b) further culturing the human pluripotent stem cells of (a) in hypoxia conditions for about 48 hours in serum-free culture medium comprising bone morphogenetic protein 4 (BMP4), FGF2, Activin A, an inhibitor of glycogen synthase 3 (GSK3), and a ROCK inhibitor to induce mesoderm formation;
(c) further culturing the cultured cells of (b) in hypoxic conditions for about 48 hours in serum-free culture medium comprising FGF2, a vascular endothelium growth factor (VEGF), and an inhibitor of TGFβ-mediated signaling to induce hemogenic endothelium formation;
(d) further culturing the cultured cells of (c) in normoxic conditions for about 6 days in serum-free culture medium comprising FGF2, a VEGF, stem cell factor (SCF), thrombopoietin (TPO), interleukin-6 (IL-6), and interleukin-3 (IL-3), wherein the hemogenic endothelium differentiate into hematopoietic precursor cells (HPCs);
(e) culturing the HPCs of (d) in normoxic conditions for about 6 days in serum-free culture medium comprising macrophage colony-stimulating factor (M-CSF), IL-3, and IL-6 to obtain myeloid progenitors and monocytic cells; and
(f) further culturing the cultured cells of (e) in normoxic conditions for about 4 days in serum-free culture medium comprising M-CSF, whereby the cultured myeloid progenitors and monocytes differentiate into a cell population comprising modified macrophages.

7. The method of claim 6, wherein the inhibitor of TGFβ-mediated signaling is SB431542.

8. The method of claim 6, wherein the inhibitor of GSK3 is lithium chloride (LiCl).

9. The method of claim 6, wherein the ROCK inhibitor is Y-27632.

10. The method of claim 6, wherein the expression of SIRPα is inhibited in the human pluripotent stem cells by gene mutation, RNA-mediated inhibition, RNA editing, DNA gene editing or base editing.

11. The method of claim 10, wherein the DNA gene editing method comprises the use of a nuclease selected from a meganuclease, zinc-finger nucleases (ZFNs), transcription activator-like effector nucleases (TALENs), and Cas enzyme.

12. The method of claim 11, wherein the nuclease is a Cas9 enzyme.

13. (canceled)

14. The method of claim 6, wherein the pluripotent stem cells are induced pluripotent stem cells.

15. (canceled)

16. The methods of claim 3 wherein modified macrophages are produced from pluripotent stem cells by a method comprising:
(a) transiently introducing exogenous ETV2 in human pluripotent stem cells having inhibited expression of SIRPα and culturing the ETV2-induced pluripotent stem cells in serum-free culture medium comprising FGF-2 to produce a population of ETV2-induced hematoendothelial progenitor cells (ETV2-induced HEPs);
(b) culturing the ETV2-induced HEPs in serum-free and xeno-free culture medium comprising granulocyte-macrophage colony-stimulating factor (GM-CSF) and FGF2 for a sufficient time to produce non-adherent myeloid progenitors;
(c) culturing the non-adherent myeloid progenitors in serum-free and xeno-free culture medium comprising M-CSF, IL-6, and IL-3; and
(d) further culturing the cultured cells of (c) in serum-free and xeno-free culture medium comprising M-CSF for a sufficient time to differentiate the non-adherent myeloid progenitors into modified macrophages.

17. The method of claim 16, wherein the serum-free and xeno-free culture medium in (b) further comprises UM171.

18. The method of claim 16, wherein the expression of SIRPα is inhibited in the human pluripotent stem cells by gene mutation, RNA-mediated inhibition, RNA editing, DNA gene editing or base editing.

19. The method of claim 18, wherein the gene editing method comprises the use of a nuclease selected from a meganuclease, ZFNs, TALENs, and Cas enzyme.

20. The method of claim 18, wherein the nuclease is a Cas9 enzyme.

21. (canceled)

22. The method of claim 16, wherein the method comprises one or more of the following:
step (a) comprises culturing the ETV2-induced pluripotent stem cells for about 1 to 2 days;
step (b) comprises culturing the ETV2-induced HEPs for about 6 to 7 days; and
steps (c) and (d) comprise culturing the myeloid progenitor cells for about 9 to 10 days.

23-24. (canceled)

25. A homogenous population of modified macrophages, wherein the modified macrophages are SIRPA knockout macrophages with a tumor-killing capacity phenotype exhibiting heightened capacity for antibody-dependent and CAR-dependent cellular phagocytosis without experiencing hypophagia.

26. A method of treating cancer comprising administering to a patient in need thereof a therapeutically effective amount of the modified macrophages of claim 25.

27. The method of claim 3 wherein the SIRPA knockout macrophages express an anti-disialoganglioside GD2 (GD2) chimeric antigen receptor (CAR).

28. (canceled)

29. The method of claim 3 wherein the tumor cell comprises a neuroblastoma, retinoblastoma, medulloblastoma, glioblastoma, melanoma, lung cancer, pancreatic cancer bladder cancer, colorectal cancer, sarcoma, or breast cancer cell.

30. (canceled)
